(19) United States
(12) Patent Application Publication
(10) Pub. No.: US 2005/0038304 A1
(43) Pub. Date: Feb. 17, 2005

(54) Integrating a methanol to olefin reaction system with a steam cracking system

(76) Inventors: Cor F. Van Egmond, Pasadena, TX (US); David Jon Duhon, Kingwood, TX (US); Jaimes Sher, Houston, TX (US)

Correspondence Address: EXXONMOBIL CHEMICAL COMPANY, 5200 BAYWAY DRIVE, P.O. BOX 2149, BAYTOWN, TX, 77522-2149, US

(21) Appl. No.: 10/642,074

(22) Filed: Aug. 15, 2003

(51) Int. Cl. C07C 1/00

(52) U.S. Cl. 585/324; 422/131; 422/188

(57) ABSTRACT

The present invention provides an integrated system for producing ethylene and propylene from an oxygenate to olefin (OTO) reaction system and a steam cracking system. In a preferred embodiment, at least a portion of an effluent stream from a steam cracking furnace is combined with at least a portion of an effluent stream from an OTO reaction system. Preferably the combined effluent stream is processed by one or more quench units, compression units, and/or fractionation columns. By integrating a steam cracking system with an OTO reaction system, equipment count can be reduced at a significant commercial savings. Compressor efficiency per pound of ethylene and propylene can also be advantageously increased over conventional steam cracking systems. Moreover, the amount of pollutants produced per pound of ethylene and propylene produced can be significantly reduced over the amount of pollutants produced per pound of ethylene and propylene produced in a steam cracking system.

### FIELD OF THE INVENTION

The present invention relates to integrated systems for producing ethylene and propylene. More particularly, the present invention relates to integrating a methanol to olefin reaction system with a steam cracking system.

### BACKGROUND OF THE INVENTION

Light olefin such has ethylene and propylene are important commodity petrochemicals useful in a variety of processes for making plastics and other chemical compounds. Ethylene is used to make various polyethylene plastics, and in making other chemicals vinyl chloride, ethylene oxide, ethyl benzene and alcohol. Propylene is used to make various polypropylene plastics, and in making other chemicals such as acrylonitrile and propylene oxide.

The petrochemical industry has known for some time that oxygenates, especially alcohols, are convertible into light olefins. The preferred conversion process is generally referred to as an oxygenate to olefin (OTO) reaction process. When methanol is the oxygenate, the process is specifically referred to as a methanol to olefin (MTO) reaction process. Methanol is a particularly preferred oxygenate for the synthesis of ethylene and/or propylene. Specifically, in an OTO reaction process, an oxygenate contacts a molecular sieve catalyst composition under the conditions effective to convert at least a portion of the oxygenate to light olefins.

Traditionally, ethylene and propylene has been produced on a commercial scale through steam cracking. In steam cracking, hydrocarbons are thermally cracked in the presence of steam to produced lighter products, particularly ethylene and propylene. Typical steam cracking feedstocks range from gaseous paraffins to naphtha and gas oils. Specifically, in steam cracking, the hydrocarbons are pyrolyzed in the presence of steam in tubular metal coils within furnaces. Steam acts as a diluent and the hydrocarbon cracks to produce olefins, diolefins, and other byproducts. Typical steam cracking processes are described, for example, in U.S. Pat. Nos. 3,365,387 and 4,061,562, the entireties of which are incorporated herein by reference.

In steam cracking, the hydrocarbon feedstock is not only converted to ethylene and propylene but also forms many low value or otherwise undesirable byproducts. These undesirable byproducts add loads to existing process equipment. Light products such as hydrogen and methane add loads to the compression and refrigeration trains. Heavier products such as gasoline, gas oils and fuel oils add loads to effluent quench systems. These added loads limit the ability for the processing equipment in a steam cracking system to recover additional ethylene and propylene production. That is, in steam cracking systems, the additional loads created by processing undesired byproducts limits the amount of ethylene and propylene that can be produced. Thus, the need exists for a process and system that can improve production of ethylene and propylene without adding excessive loads to existing compression and fractionation equipment.

An additional problem associated with steam cracking systems is that as additional cracking furnaces are added to a steam cracking system to produce more light olefins, air pollutant production, particularly NOX and greenhouse gas production (e.g., CO2 production) increases. Thus, the need also exists for reducing the amount of air pollutants produced per pound of ethylene and propylene in a steam cracking system.

### SUMMARY OF THE INVENTION

The present invention provides for the integration of an oxygenate to olefin (OTO) reaction system with a steam cracking system. In a preferred embodiment, at least a portion of an effluent stream from a steam cracking furnace is combined with at least a portion of an effluent stream from an OTO reaction system. Preferably, the combined effluent stream is processed by one or more quench units, compression units, and/or fractionation columns. By integrating a steam cracking system with an OTO reaction system, equipment count can be reduced at a significant commercial savings. Additionally, the amount of pollutants produced per pound of ethylene and propylene produced can be significantly reduced over the amount of pollutants produced per pound of ethylene and propylene produced in a steam cracking system.

In one embodiment, the invention is to a process for forming light olefins. In the process, hydrocarbons contact steam in a cracking furnace under first conditions effective to form a first effluent stream containing fuel oil, gas oil, gasoline and light olefins. Simultaneously, methanol contacts a molecular sieve catalyst in a methanol-to-olefin reactor under second conditions effective to form a second effluent stream containing light olefins, C4+ hydrocarbons and water. At least a portion of the first effluent stream and at least a portion of the second effluent stream are combined to form a combined stream. The combined stream contacts a quench medium in a quench unit under conditions effective to form a quench overhead stream and a quench bottoms stream, wherein the quench overhead stream contains at least a majority of the light olefins that were present in the combined stream, and wherein the quench bottoms stream contains at least a portion of the water and condensed components. At least a portion of the quench overhead stream is fractionated to form a light olefins product stream and one or more C4+ hydrocarbon streams, wherein the light olefins product stream contains a majority of the light olefins that were present in the at least a portion of the quench overhead stream. The light olefins product stream optionally contains at least 50, at least 75 or at least 90 weight percent light olefins, based on the total weight of the light olefins product stream.

In another embodiment, hydrocarbons contact steam in a cracking furnace under first conditions effective to form a first effluent stream containing fuel oil, gas oil, gasoline and light olefins. The first effluent stream is separated into a light olefins-containing stream and one or more heavy hydrocarbon streams, wherein the light olefins-containing stream contains at least a majority of the light olefins that were present in the first effluent stream. Simultaneously, methanol contacts a molecular sieve catalyst in a methanol-to-olefin reactor under second conditions effective to form a second effluent stream containing light olefins, C4+ hydrocarbons and water. At least a portion of the light olefins-containing stream and at least a portion of the second effluent stream are combined to form a combined stream. The combined stream contacts a quench medium in a quench unit under conditions effective to form a quench overhead stream and a quench bottoms stream, wherein the quench overhead stream contains at least a majority of the light olefins that were present in the combined stream, and wherein the quench bottoms stream contains at least a portion of the water and condensed components. At least a portion of the quench overhead stream is fractionated to form a light olefins product stream and one or more C4+ hydrocarbon streams, wherein the light olefins product stream contains a majority of the light olefins that were present in the at least a portion of the quench overhead stream.

In another embodiment, the steam cracking and OTO reaction systems are integrated after product quenching. In this embodiment, hydrocarbons contact steam in a cracking furnace under first conditions effective to form a first effluent stream containing fuel oil, gas oil, gasoline, water and light olefins. The first effluent stream is separated into a light olefins-containing stream and one or more heavy hydrocarbon streams, wherein the light olefins-containing stream contains at least a majority of the light olefins that were present in the first effluent stream. At least a portion of the light olefins-containing stream contacts a first quench medium in a first quench unit under second conditions effective to form a first quench overhead stream and a first quench bottoms stream, wherein the first quench overhead stream contains at least a majority of the light olefins that were present in the at least a portion of the light olefins-containing stream, and wherein the first quench bottoms stream contains first condensed components and at least a majority of the water that was present in the at least a portion of the light olefins-containing stream. Simultaneously, methanol contacts a molecular sieve catalyst in a methanol-to-olefin reactor under third conditions effective to form a second effluent stream containing water and light olefins. At least a portion of the second effluent stream contacts a second quench medium in a second quench unit under fourth conditions effective to form a second quench overhead stream and a second quench bottoms stream, wherein the second quench overhead stream contains at least a majority of the light olefins that were present in the at least a portion of the second effluent stream, and wherein the second quench bottoms stream contains second condensed components and at least a majority of the water that was present in the at least a portion of the second effluent stream. At least a portion of the first quench overhead stream is combined with at least a portion of the second quench overhead stream to form a combined stream. At least a portion of the combined stream is compressed to form a compressed stream, and at least a portion of the compressed stream is fractionated to form a light olefins product stream and one or more C4+ hydrocarbon streams, wherein the light olefins product stream contains a majority of the light olefins that were present in the compressed stream.

In another embodiment, the steam cracking and OTO reaction systems are integrated after effluent compression. In this embodiment, hydrocarbons contact steam in a cracking furnace under first conditions effective to form a first effluent stream containing fuel oil, gas oil, gasoline and light olefins. The first effluent stream is separated into a light olefins-containing stream and one or more heavy hydrocarbon streams, wherein the light olefins-containing stream contains at least a majority of the light olefins that were present in the first effluent stream. At least a portion of the light olefins-containing stream contacts a first quench medium in a first quench unit under second conditions effective to form a first quench overhead stream and a first quench bottoms stream, wherein the first quench overhead stream contains at least a majority of the light olefins that were present in the at least a portion of the light olefins-containing stream, and wherein the first quench bottoms stream contains first condensed components and at least a majority of the water that was present in the at least a portion of the light olefins-containing stream. At least a portion of the first quench overhead stream is compressed to form a first compressed stream. Simultaneously, methanol contacts a molecular sieve catalyst in a methanol-to-olefin reactor under third conditions effective to form a second effluent stream containing water and light olefins. At least a portion of the second effluent stream contacts a second quench medium in a second quench unit under fourth conditions effective to form a second quench overhead stream and a second quench bottoms stream, wherein the second quench overhead stream contains at least a majority of the light olefins that were present in the at least a portion of the second effluent stream, and wherein the second quench bottoms stream contains second condensed components and at least a majority of the water that was present in the at least a portion of the second effluent stream. At least a portion of the second quench overhead stream is compressed to form a second compressed stream. At least a portion of the first compressed stream and at least a portion of the second compressed stream are combined to form a combined stream, and at least a portion of the combined stream is fractionated to form a light olefins product stream and one or more C4+ hydrocarbon streams, wherein the light olefins product stream contains a majority of the light olefins that were present in the combined stream.

The invention is also directed to integrated systems for forming light olefins. In one embodiment, the system comprises a steam cracking furnace having one or more inlets for receiving hydrocarbons and steam, and a first outlet line for carrying a first product stream. The system also includes a methanol to olefin conversion unit having one or more inlets for receiving a methanol-containing feedstock, and a second outlet line for carrying a second product stream. A quench unit is in fluid communication with the first outlet line and the second outlet line, wherein the quench unit includes an overhead line, a condensate line and a pumparound stream, wherein the pumparound stream is arranged to deliver condensed components from a lower region of the quench unit to an upper region of the quench unit. A compressor is also included having a compressor inlet and a compressor outlet, wherein the compressor inlet is in fluid communication with the overhead line. The system also includes a fractionation system having a fractionator inlet, a light olefin outlet line and one or more C4+ hydrocarbon outlet lines, wherein the fractionator inlet is in fluid communication with the compressor outlet.

In another embodiment, the invention is to a system for forming light olefins, the system including a steam cracking furnace having one or more inlets for receiving hydrocarbons and steam, and a first outlet line for carrying a first product stream. The system also includes a first quench unit in fluid communication with the first outlet line, wherein the first quench unit includes a first overhead line, a first condensate line and a first pumparound stream, wherein the first pumparound stream is arranged to deliver first condensed components from a first lower region of the first quench unit to a first upper region of the first quench unit. Also, the system comprises a methanol to olefin conversion unit having one or more inlets for receiving a methanol-containing feedstock, and a second outlet line for carrying a second product stream. A second quench unit is in fluid communication with the second outlet line, wherein the second quench unit includes a second overhead line, a second condensate line and a second pumparound stream, wherein the second pumparound stream is arranged to deliver second condensed components from a second lower region of the second quench unit to a second upper region of the second quench unit. A compressor is also provided having a compressor inlet and a compressor outlet, wherein the compressor inlet is in fluid communication with the first overhead line and the second overhead line. The system also includes a fractionation system having a fractionator inlet, a light olefin outlet line and one or more C4+ hydrocarbon outlet lines, wherein the fractionator inlet is in fluid communication with the compressor outlet.

In another embodiment, the system comprises a steam cracking furnace having one or more inlets for receiving hydrocarbons and steam, and a first outlet line for carrying a first product stream. A first quench unit is provided in fluid communication with the first outlet line, wherein the first quench unit includes a first overhead line, a first condensate line and a first pumparound stream, wherein the first pumparound stream is arranged to deliver first condensed components from a first lower region of the first quench unit to a first upper region of the first quench unit. A first compressor having a first compressor inlet and a first compressor outlet is also provided, wherein the first compressor inlet is in fluid communication with the first overhead line. The system also includes a methanol to olefin conversion unit having one or more inlets for receiving a methanol-containing feedstock, and a second outlet line for carrying a second product stream. A second quench unit is in fluid communication with the second outlet line, wherein the second quench unit includes a second overhead line, a second condensate line and a second pumparound stream, wherein the second pumparound stream is arranged to deliver second condensed components from a second lower region of the second quench unit to a second upper region of the second quench unit. Also, the invention includes a second compressor having a second compressor inlet and a second compressor outlet, wherein the second compressor inlet is in fluid communication with the second overhead line. The system also includes a fractionation system having a fractionator inlet, a light olefin outlet line and one or more C4+ hydrocarbon outlet lines, wherein the fractionator inlet is in fluid communication with the first compressor outlet and the second compressor outlet.

### BRIEF DESCRIPTION OF THE DRAWINGS

This invention will be better understood by reference to the detailed description of the invention when taken together with the attached drawings, wherein:

FIG. 1 illustrates a stream cracking system;

FIG. 2 illustrates an oxygenate to olefin reaction system;

FIG. 3 illustrates an integrated steam cracking and oxygenate to olefin reaction system, wherein the integrated system shares a quench unit, a compression unit, and one or more separation units;

FIG. 4 illustrates an integrated steam cracking system and oxygenate to olefin reaction system, wherein the integrated system shares a compression unit and one or more separation units; and

FIG. 5 illustrates an integrated steam cracking system and oxygenate to olefin reaction system, wherein the integrated system shares one or more separation units.

### DETAILED DESCRIPTION OF THE INVENTION

Introduction

The present invention provides an integrated reaction system for producing ethylene and propylene. Specifically, the integrated system according to the present invention combines an effluent from a steam cracking system with an effluent from an oxygenate to olefin (OTO) reaction system, preferably a methanol to olefin (MTO) reaction system. By combining effluent streams from a steam cracking system and an OTO reaction system, equipment count in the integrated system can be advantageously minimized. Also, compressor efficiency per pound of ethylene and propylene produced can be advantageously increased over compressor systems in conventional steam cracking systems. Additionally, the amount of pollutants produced per pound of ethylene and propylene produced can be significantly lower than in a conventional steam cracking system.

Steam Cracking Systems

The bulk of ethylene consumed in the production of various plastics and petrochemicals such as polyethylene is produced by thermally cracking higher molecular weight hydrocarbons in a feed stream. Steam is usually admixed with the feed stream that is directed to the cracking reactor to reduce the hydrocarbon partial pressure and thereby enhance olefin yield. The presence of steam also reduces the formation and deposition of carbonaceous material in the cracking reactors. The process is therefore often referred to as steam cracking or pyrolysis. The feedstock that is fed to a steam cracking unit can be quite diverse and can be chosen from a variety of petroleum fractions.

The feed stream or feedstock that is directed to a steam cracking unit preferably has a boiling point range falling within the naphtha boiling point range of about 36° C. to about 195° C. Naphtha is a gasoline range boiling hydrocarbon having a carbon range of C5 to C12. Optionally, the feed stream that is directed to the steam cracking unit comprises C2 to C40 hydrocarbons, more preferably C2 to C30 hydrocarbons (e.g., including naphtha, field condensates, various gas oil cuts and crude oil), and most preferably C2 to C20 hydrocarbons. In another embodiment, the stream cracking feed comprises C2 to C4 hydrocarbons. In this embodiment, the stream cracking feed comprises one or more of ethane, propane, ethane/propane mixed, or a mixed C4 stream.

As used herein, “light olefins” means ethylene and propylene. “Fuel oil” is a hydrocarbon-containing composition, in any physical state, comprising mostly C9+ hydrocarbons; “gas oil” is a hydrocarbon-containing composition, in any physical state, comprising mostly C7-C8 hydrocarbons; and “gasoline” is a hydrocarbon-containing composition, in any physical state, comprising mostly C4-C6 hydrocarbons.

Within the steam cracking unit, the feed stream contacts steam under conditions, e.g., temperature and pressure, effective to convert at least a portion of the feed stream to ethylene and propylene, which exits the steam cracking unit in a steam cracking effluent stream. The steam cracking effluent stream contains a variety of contaminants and C4+ components in addition to ethylene and propylene, and separation of these various hydrocarbon components is necessary in order to yield chemical or polymerization grade ethylene and propylene. For example, upon exiting the steam cracking unit, the steam cracking effluent stream preferably is cooled in an indirect quench unit to form a cooled steam cracking effluent stream, which is preferably directed to one or more fractionation units.

Ideally, the one or more fractionation units comprises a fractionation column capable of separating the cooled steam cracking effluent stream into two or more derivative streams. As used herein, “fractionating” means separating a stream, e.g., by distillation, into two or more non-aliquot derivative streams. Preferably, the one or more fractionation units separates the cooled steam cracking effluent stream into one or more of: a light hydrocarbon stream containing mostly C5− components, a gasoline stream (also referred to as pyrolysis gasoline or pygas) containing mostly C6 components and optionally water, a gas oil stream containing C7 to C8 hydrocarbons, a fuel oil stream containing C9+ hydrocarbon components, and a coke removal stream for removing carbonaceous deposits from the one or more fractionation units. Specifically, the fuel oil stream contains at least a majority of the fuel oil that was present in the cooled steam cracking effluent stream. The gas oil stream contains at least a majority of the gas oil that was present in the cooled steam cracking effluent stream. The gasoline stream contains at least a majority of the gasoline that was present in the cooled steam cracking effluent stream.

Ideally, the light hydrocarbon stream (optionally comprising a minor amount of C6 components) is directed from the one or more fractionation units to a quench tower for direct light hydrocarbon stream quenching. In the quench tower, the light hydrocarbon stream, or a portion thereof, contacts a quench medium, preferably water, under conditions effective to separate readily condensable components from non-readily condensable components. Preferably, two phases form in the bottom of the quench tower, a pygas stream which contains mostly C6 hydrocarbons, and a heavier water containing stream. A portion of the water containing stream optionally is cooled in one or more heat exchangers and reintroduced into the quench tower via one or more quench medium inlets. Thus, in this embodiment, condensed water from the light hydrocarbon stream serves as the quench medium in the quench tower. Nonreadily condensable components such as ethylene and propylene are withdrawn from the quench tower via a quench overhead stream. The majority of the C5-components preferably are yielded from the quench tower in the quench overhead stream.

The quench overhead stream from the quench tower preferably is directed to a compression system containing one or more compression stages, preferably at least four compression stages. In a preferred embodiment, the compression system comprises one or more centrifugal compressors, one or more heat exchangers for cooling intermediate condensed streams, and one or more knockout drums to separate condensed components from noncondensed components. The compression system preferably forms a compressed effluent stream, which is ideally directed to a caustic wash unit for removal of carbon dioxide therefrom.

Specifically, in the caustic wash unit, the compressed stream contacts caustic, e.g., sodium hydroxide, under conditions effective to remove carbon dioxide therefrom. Sodium bicarbonate (NaHCO3) optionally is formed as a byproduct of the carbon dioxide removal process. Thus, the caustic wash unit forms an overhead CO2 depleted stream and a spent caustic bottoms stream. Optionally, the CO2 depleted stream or a portion thereof is compressed in one or more additional stages. Also, the CO2 depleted stream or a portion thereof optionally is directed to a drying unit, preferably a molecular sieve drying unit, wherein the CO2 depleted stream contacts a water removal medium under conditions effective to remove water from the CO2 depleted stream. Preferably, the water removal medium comprises a molecular sieve particle adapted to selectively adsorb water molecules. That is, the drying unit removes water from the carbon dioxide depleted stream to form a dry product stream comprising ethylene, propylene and optionally light ends such as hydrogen, carbon monoxide and methane and/or C4+ hydrocarbons. The dry product stream, or a portion thereof, is then directed to a separation system that is adapted to separate the dry stream into two or more of its individual components.

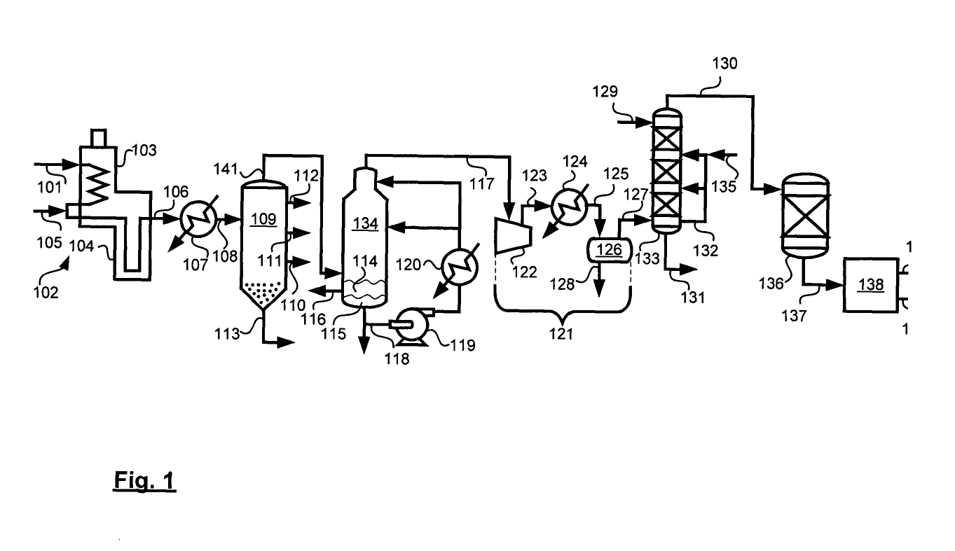

FIG. 1 illustrates a steam cracking system. As shown, a C2 to C40 containing feedstock 101 as directed to steam cracking unit 102. Preferably, feedstock 101 comprises C2 to C30 hydrocarbons, and more preferably C2 to C20 hydrocarbons. Steam cracking unit 102 preferably comprises a preheat region 103 and a cracking region 104. In the preheat region 103, the feedstock 101 is heated to form a heated feedstock stream having a temperature of from about 200° F. (93° C.) to about 450° F. (232° C.). Steam from steam containing line 105 preferably is admixed with the heated feedstock 101, which is then directed to cracking region 104. In cracking region 104, the steam containing feedstock 101 is further heated under conditions effective to “crack” the hydrocarbons and produce an effluent stream 106 comprising fuel oil, gas oil, pyrolysis gasoline, and C5− hydrocarbon components (including ethylene and propylene).

Effluent stream 106 optionally is directed to indirect quench unit 107 for indirect effluent stream quenching. In the indirect quench unit 107, effluent stream 106 is cooled under conditions effective to form cooled stream 108. Cooled stream 108 is then preferably directed to one or more fractionation units 109. In fractionation unit 109, cooled stream 108 is subjected to temperature and pressure conditions that are effective to separate the cooled stream 108 into a plurality of derivative non-aliquot streams. As shown, cooled stream 108 is fractionated in fractionation unit 109 to form fuel oil stream 110, gas oil stream 111, gasoline stream 112, and light hydrocarbon stream 141 (containing C5− hydrocarbons including ethylene and propylene).

Carbonaceous deposits (coke) are formed as a byproduct of the steam cracking process. These carbonaceous deposits exit the steam cracking unit 102 via effluent stream 106 and are directed through indirect quench unit 107 and cooled stream 108 to fractionation unit 109. Upon introduction into fractionation unit 109, the carbonaceous deposits settle and are separated from the liquid components contained in fractionation unit 109. The settled carbonaceous deposits in fractionation unit 109 ideally are withdrawn therefrom, as shown by coke removal stream 113.

Light hydrocarbon stream 141 preferably is then directed to quench unit 134, wherein readily condensable components in light hydrocarbon stream 141 are condensed from nonreadily condensable components contained therein. Specifically, in quench unit 134, light hydrocarbon stream 141 or a portion thereof contacts a quenching medium under conditions effective to condense out the readily condensable components contained therein. The readily condensable components in light hydrocarbon stream 141 comprise aqueous and hydrocarbon components, which separate in the quench unit 134 and form two condensed phases, specifically, hydrocarbon phase 114 and aqueous phase 115.

As shown, hydrocarbon phase 114 contains mostly C6 hydrocarbon components, which are withdrawn from quench unit 134 via pygas stream 116. Aqueous phase 115 is withdrawn from quench unit 134 via condensate stream 118 with the assistance of pump 119. Pump 119 directs a portion of condensate stream 118 through heat exchanger 120, which cools condensate stream 118. The cooled condensate stream 118 is then directed back to quench unit 134 wherein the cooled condensate stream 118 serves as a quench medium to condense out readily condensable components that enter quench unit 134 via light hydrocarbon stream 141. Optionally, cooled condensate stream 118 is separated into a plurality of streams, which introduce the cooled condensate stream 118 into quench unit 134 through a plurality of inlets in quench unit 134. Non-readily condensable components contained in light hydrocarbon stream 141 exit quench unit 134 via quench overhead stream 117.

Quench overhead stream 117 ideally is then compressed in one or more compression stages, preferably a plurality of compression stages. For clarity, FIG. 1 illustrates a single compression stage 121, which comprises a compressor 122, a heat exchanger 124 and a knockout drum 126. However, the compression system optionally comprises two, three, four or more compression stages. Reverting to FIG. 1, quench overhead stream 117 is directed to compressor 122, which compresses quench overhead stream 117 and forms compressed stream 123. Compressed stream 123 is then directed to a heat exchanger 124 in which the compressed stream 123 indirectly contacts a cooling medium under conditions effective to cool compressed stream 123 and form cooled compressed stream 125. Cooled compressed stream 125 is then directed to a knockout drum 126, wherein readily condensable components are allowed to condense from non-readily condensable components. The readily condensable components exit knockout drum 126 via condensed stream 128. If the readily condensable components contained in cooled compressed stream 125 comprise aqueous and hydrocarbon components, then the knockout drum 126 optionally is a two-phase or three-phase knockout drum, not shown, which is adapted to form an aqueous condensed stream, a hydrocarbon condensed stream, and a vapor stream. As shown, knockout drum 126 is adapted to separate condensed components in condensed stream 128 from gaseous components, which exit knockout drum 126 via vapor stream 127.

Vapor stream 127 ideally is then directed to a caustic wash unit 133 for removal of entrained acid gases such as CO2. That is, caustic wash unit 133 removes CO2 and other entrained acid gases from the vapor stream 127, or a portion thereof, and forms a CO2 depleted stream 130. The vapor stream 127 preferably contains relatively few hydrocarbon components that cause fouling problems in such acid gas treatment systems.

Solid or liquid acid gas treatment systems can be used in this invention. In either system, the acid gas is removed from the wash overhead stream or a stream derived therefrom by contacting the stream with an acid gas absorbent or adsorbent. Examples of such absorbents or adsorbents include amines, potassium carbonate, caustic, alumina, molecular sieves, and membranes, particularly membranes formed of polysulfone, polyimid, polyamide, glassy polymer and cellulose acetate. Solutions containing amines and caustic compounds are preferred, with caustic compounds being more preferred.

Aqueous amine solutions that are useful in this invention can contain any amine compound or compounds suitable for acid gas absorption. Examples include alkanolamines, such as triethanolamine (TEA); methyldiethanolamine (MDEA); diethanolamine (DEA); monoethanolamine (MEA); diisopropanolamine (DIPA); and hydroxyaminoethyl ether (DGA). Effective concentrations can range from about 0.5 to about 8 moles of amine per liter of aqueous solution.

Piperazine and/or monomethylethanolamine (MMEA) can be added to aqueous amine solutions to enhance their absorption capabilities. These additives can be included in the aqueous solution at a concentration of from about 0.04 to about 2 moles per liter of aqueous solution.

Caustic compounds that can be used in this invention are alkaline compounds, which are effective in removing acid gas from an initial effluent stream. Examples of such alkaline compounds include sodium hydroxide and potassium hydroxide.

Reverting to FIG. 1, caustic wash unit 133 comprises one or more, preferably a plurality of beds (three are shown) and a pumparound stream 132. Pumparound stream 132 withdraws a portion of the spent caustic from caustic wash unit 133 and recirculates it to one or more higher portions on the caustic wash unit 133. As shown, pumparound stream 132 withdraws a portion of the spent caustic solution from the caustic wash unit 133 and recirculates it into caustic wash unit 133 through two inlets. Fresh caustic 135 optionally is introduced into the pumparound stream 132 or directly into caustic wash unit 133. Optionally, water stream 129 is also introduced into caustic wash unit 133, preferably adjacent the top end thereof, in order to ensure that CO2 depleted stream 130 does not contain entrained caustic medium, or contains a minimal amount of caustic medium. In this manner, caustic wash unit 133 functions to remove CO2 and other entrained acid gas components as well as functions as a water wash unit. Thus, upon introduction into caustic wash unit 133, vapor stream 127 contacts a caustic medium 135 under conditions effective to remove CO2 and other entrained acid gas components thereof, and form CO2 depleted stream 130 and spent caustic stream 131. CO2 depleted stream 130 is then optionally directed to a drying unit 136. Optionally, CO2 depleted stream 130 is compressed in one or more additional stages, not shown, between caustic wash unit 133 and drying unit 136.

In this embodiment, a solid or liquid drying system can be used to remove water and/or additional oxygenated hydrocarbons from the effluent stream that is directed thereto, e.g., CO2 depleted stream 130. In the solid drying system, the effluent stream, e.g., CO2 depleted stream 130, is received in a drying unit. In the drying unit, the effluent stream contacts a solid adsorbent to further remove water and oxygenated hydrocarbons to very low levels. Typically, the adsorption process is carried out in one or more fixed beds containing a suitable solid adsorbent.

Adsorption is useful for removing water and oxygenated hydrocarbons to very low concentrations, and for removing oxygenated hydrocarbons that are not normally removed by using other treatment systems. Preferably, an adsorbent system used as part of this invention has multiple adsorbent beds. Multiple beds allow for continuous separation without the need for shutting down the process to regenerate the solid adsorbent. For example, in a three bed system typically one bed is on-line, one bed is regenerated off-line, and a third bed is on stand-by.

The specific adsorbent solid or solids used in the adsorbent beds depends on the types of contaminants being removed. Examples of solid adsorbents for removing water and various polar organic compounds, such as oxygenated hydrocarbons and absorbent liquids, include aluminas, silica, 3Å molecular sieves, 4Å molecular sieves, and alumino-silicates. Beds containing mixtures of these sieves or multiple beds having different adsorbent solids can be used to remove water, as well as a variety of oxygenated hydrocarbons.

In this separation technique, one or more adsorption beds can be arranged in series or parallel. In one example of a series arrangement, a first bed is used to remove the smallest and most polar molecules, which are the easiest to remove. Subsequent beds for removing larger less polar oxygenated species are next in series. As a specific example of one type of arrangement, water is first selectively removed using a 3Å molecular sieve. This bed is then followed by one or more beds containing one or more less selective adsorbents such as a larger pore molecular sieve, e.g., 13× and/or a high surface area active alumina such as Selexorb CD (Alcoa tradename).

In another embodiment, the first bed is a 3.6Å molecular sieve capable of selectively removing both water and methanol. This bed can then be followed by one or more 13× or active alumina beds as described above.

The adsorbent beds can be operated at ambient temperature or at elevated temperature as required, and with either upward or downward flow. Regeneration of the adsorbent materials can be carried out by conventional methods including treatment with a stream of a dry inert gas such as nitrogen at elevated temperature.

In the liquid drying system, a water absorbent is used to remove water from the effluent stream received therein. The water absorbent can be any liquid effective in removing water from an olefin-containing stream. Preferably, the water absorbent is the same as that previously described.

The drying system of the present invention, whether employing a liquid or a solid drying agent, ideally forms a “dry stream,” defined herein as a stream having a dew point of less than −40° F. (−40° C.). Preferably the dry stream from the drying unit has a dew point of less than −40° F. (−40° C.), more preferably less than about −60° F. (−51 ° C.), and most preferably less than −90° F. (−67.8° C.). The dry stream preferably is then directed to a separation system for removal of the remaining components contained therein, as described in more detail below.

As shown in FIG. 1, CO2 depleted stream 130 contacts a drying agent in drying unit 136 under conditions effective to form dry stream 137. Dry stream 137 is then directed to a separation system 138, in which the components of dry stream 137 ideally are separated into two or more non-aliquot product streams. As shown, separation system 138 separates dry stream 137 into ethylene-containing stream 139 and propylene-containing stream 140. It is more likely, however, that separation system 138 separates the dry stream 137 into more than two derivative non-aliquot streams. Separation system 138 preferably comprises one or more separation units, preferably a plurality thereof. The separation units optionally include one or more distillation columns, splitters, separators, wash columns, absorption units, or other separation devices.

Oxygenate to Olefin Reaction Systems

Typically, molecular sieve catalysts have been used to convert oxygenate compounds to light olefins. Silicoaluminophosphate (SAPO) molecular sieve catalysts are particularly desirable in such a conversion process, because they are highly selective in the formation of ethylene and propylene. A non-limiting list of preferable SAPO molecular sieve catalysts includes SAPO-17, SAPO-18, SAPO-34, SAPO-35, SAPO-44, the substituted forms thereof, and mixtures thereof.

The feedstock preferably contains one or more aliphatic-containing compounds that include alcohols, amines, carbonyl compounds for example aldehydes, ketones and carboxylic acids, ethers, halides, mercaptans, sulfides, and the like, and mixtures thereof. The aliphatic moiety of the aliphatic-containing compounds typically contains from 1 to about 50 carbon atoms, preferably from 1 to 20 carbon atoms, more preferably from 1 to 10 carbon atoms, and most preferably from 1 to 4 carbon atoms.

Non-limiting examples of aliphatic-containing compounds include: alcohols such as methanol and ethanol, alkyl-mercaptans such as methyl mercaptan and ethyl mercaptan, alkyl-sulfides such as methyl sulfide, alkyl-amines such as methyl amine, alkyl-ethers such as DME, diethyl ether and methylethyl ether, alkyl-halides such as methyl chloride and ethyl chloride, alkyl ketones such as dimethyl ketone, alkyl-aldehydes such as formaldehyde and acetaldehyde, and various acids such as acetic acid.

In a preferred embodiment of the process of the invention, the feedstock contains one or more oxygenates, more specifically, one or more organic compound(s) containing at least one oxygen atom. In the most preferred embodiment of the process of invention, the oxygenate in the feedstock is one or more alcohol(s), preferably aliphatic alcohol(s) where the aliphatic moiety of the alcohol(s) has from 1 to 20 carbon atoms, preferably from 1 to 10 carbon atoms, and most preferably from 1 to 4 carbon atoms. The alcohols useful as feedstock in the process of the invention include lower straight and branched chain aliphatic alcohols and their unsaturated counterparts. Non-limiting examples of oxygenates include methanol, ethanol, n-propanol, isopropanol, methyl ethyl ether, DME, diethyl ether, di-isopropyl ether, formaldehyde, dimethyl carbonate, dimethyl ketone, acetic acid, and mixtures thereof. In the most preferred embodiment, the feedstock is selected from one or more of methanol, ethanol, DME, diethyl ether or a combination thereof, more preferably methanol and DME, and most preferably methanol.

The various feedstocks discussed above, particularly a feedstock containing an oxygenate, more particularly a feedstock containing an alcohol, is converted primarily into one or more olefin(s). The olefin(s) or olefin monomer(s) produced from the feedstock typically have from 2 to 30 carbon atoms, preferably 2 to 8 carbon atoms, more preferably 2 to 6 carbon atoms, still more preferably 2 to 4 carbons atoms, and most preferably ethylene an/or propylene.

Non-limiting examples of olefin monomer(s) include ethylene, propylene, butene-1, pentene-1, 4-methyl-pentene-1, hexene-1, octene-1 and decene-1, preferably ethylene, propylene, butene-1, pentene-1, 4-methyl-pentene-1, hexene-1, octene- 1 and isomers thereof. Other olefin monomer(s) include unsaturated monomers, diolefins having 4 to 18 carbon atoms, conjugated or nonconjugated dienes, polyenes, vinyl monomers and cyclic olefins.

In the most preferred embodiment, the feedstock, preferably of one or more oxygenates, is converted in the presence of a molecular sieve catalyst composition into olefin(s) having 2 to 6 carbons atoms, preferably 2 to 4 carbon atoms. Most preferably, the olefin(s), alone or combination, are converted from a feedstock containing an oxygenate, preferably an alcohol, most preferably methanol, to the preferred olefin(s) ethylene and/or propylene.

The most preferred process is generally referred to as gas-to-olefins (GTO) or alternatively, methanol-to-olefins (MTO). In an MTO process, a methanol containing feedstock, is converted in the presence of a molecular sieve catalyst composition into one or more olefins, preferably and predominantly, ethylene and/or propylene, often referred to as light olefins.

The feedstock, in one embodiment, contains one or more diluents, typically used to reduce the concentration of the feedstock. The diluents are generally non-reactive to the feedstock or molecular sieve catalyst composition. Non-limiting examples of diluents include helium, argon, nitrogen, carbon monoxide, carbon dioxide, water, essentially non-reactive paraffins (especially alkanes such as methane, ethane, and propane), essentially non-reactive aromatic compounds, and mixtures thereof. The most preferred diluents are water and nitrogen, with water being particularly preferred. In other embodiments, the feedstock does not contain any diluent.

The diluent may be used either in a liquid or a vapor form, or a combination thereof. The diluent is either added directly to a feedstock entering into a reactor or added directly into a reactor, or added with a molecular sieve catalyst composition. In one embodiment, the amount of diluent in the feedstock is in the range of from about 1 to about 99 mole percent based on the total number of moles of the feedstock and diluent, preferably from about 1 to 80 mole percent, more preferably from about 5 to about 50, most preferably from about 5 to about 25. In one embodiment, other hydrocarbons are added to a feedstock either directly or indirectly, and include olefin(s), paraffin(s), aromatic(s) (see for example U.S. Pat. No. 4,677,242, addition of aromatics) or mixtures thereof, preferably propylene, butylene, pentylene, and other hydrocarbons having 4 or more carbon atoms, or mixtures thereof.

The process for converting a feedstock, especially a feedstock containing one or more oxygenates, in the presence of a molecular sieve catalyst composition of the invention, is carried out in a reaction process in a reactor, where the process is a fixed bed process, a fluidized bed process (includes a turbulent bed process), preferably a continuous fluidized bed process, and most preferably a continuous high velocity fluidized bed process.

The reaction processes can take place in a variety of catalytic reactors such as hybrid reactors that have a dense bed or fixed bed reaction zones and/or fast fluidized bed reaction zones coupled together, circulating fluidized bed reactors, riser reactors, and the like. Suitable conventional reactor types are described in for example U.S. Pat. Nos. 4,076,796, 6,287,522 (dual riser), and Fluidization Engineering, D. Kunii and O. Levenspiel, Robert E. Krieger Publishing Company, New York, N.Y. 1977, which are all herein fully incorporated by reference.

The preferred reactor type are riser reactors generally described in Riser Reactor, Fluidization and Fluid-Particle Systems, pages 48 to 59, F. A. Zenz and D. F. Othmer, Reinhold Publishing Corporation, New York, 1960, and U.S. Pat. No. 6,166,282 (fast-fluidized bed reactor), and U.S. patent application Ser. No. 09/564,613 filed May 4, 2000 (multiple riser reactor), which are all herein fully incorporated by reference.

In an embodiment, the amount of liquid feedstock fed separately or jointly with a vapor feedstock, to a reactor system is in the range of from 0.1 weight percent to about 85 weight percent, preferably from about 1 weight percent to about 75 weight percent, more preferably from about 5 weight percent to about 65 weight percent based on the total weight of the feedstock including any diluent contained therein. The liquid and vapor feedstocks are preferably the same composition, or contain varying proportions of the same or different feedstock with the same or different diluent.

The conversion temperature employed in the conversion process, specifically within the reactor system, is in the range of from about 392° F. (200° C.) to about 1832° F. (1000° C.), preferably from about 482° F. (250° C.) to about 1472° F. (800° C.), more preferably from about 482° F. (250° C.) to about 1382° F. (750° C.), yet more preferably from about 572° F. (300° C.) to about 1202° F. (650° C.), yet even more preferably from about 662° F. (350° C.) to about 1112° F. (600° C.) most preferably from about 662° F. (350° C.) to about 1022° F. (550° C.).

The conversion pressure employed in the conversion process, specifically within the reactor system, varies over a wide range including autogenous pressure. The conversion pressure is based on the partial pressure of the feedstock exclusive of any diluent therein. Typically the conversion pressure employed in the process is in the range of from about 0.1 kPaa to about 5 MPaa, preferably from about 5 kPaa to about 1 MPaa, and most preferably from about 20 kPaa to about 500 kPaa.

The weight hourly space velocity (WHSV), particularly in a process for converting a feedstock containing one or more oxygenates in the presence of a molecular sieve catalyst composition within a reaction zone, is defined as the total weight of the feedstock excluding any diluents to the reaction zone per hour per weight of molecular sieve in the molecular sieve catalyst composition in the reaction zone. The WHSV is maintained at a level sufficient to keep the catalyst composition in a fluidized state within a reactor.

Typically, the WHSV ranges from about 1 hr-1 to about 5000 hr -1, preferably from about 2 hr-1 to about 3000 hr-1, more preferably from about 5 hr-1 to about 1500 hr-1, and most preferably from about 10 hr-1 to about 1000 hr-1. In one preferred embodiment, the WHSV is greater than 20 hr-1, preferably the WHSV for conversion of a feedstock containing methanol, DME, or both, is in the range of from about 20 hr-1 to about 300 hr-1.

The superficial gas velocity (SGV) of the feedstock including diluent and reaction products within the reactor system is preferably sufficient to fluidize the molecular sieve catalyst composition within a reaction zone in the reactor. The SGV in the process, particularly within the reactor system, more particularly within the riser reactor(s), is at least about 0.1 meter per second (m/sec), preferably greater than 0.5 m/sec, more preferably greater than 1 m/sec, even more preferably greater than 2 m/sec, yet even more preferably greater than 3 m/sec, and most preferably greater than 4 m/sec. See for example U.S. patent application Ser. No. 09/708,753 filed Nov. 8, 2000 which is herein incorporated by reference.

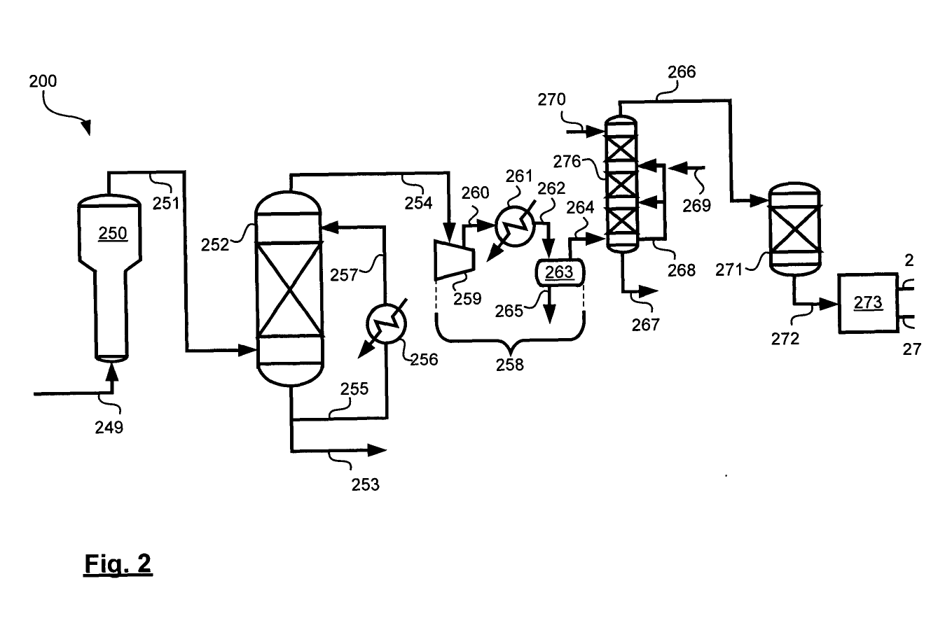

FIG. 2 illustrates an oxygenate to olefin (OTO) reaction system, generally designated 200. As shown, an oxygenate containing feedstock 249 is directed to an OTO reaction unit 250, wherein the oxygenate, e.g., methanol, in oxygenate-containing feedstock 249 contacts a molecular sieve catalyst composition under conditions effective to convert at least a portion of the oxygenate in the oxygenate-containing feedstock 249 to light olefins. The light olefins formed in reaction unit 250 are yielded therefrom in light olefins-containing stream 251.

Light olefins-containing stream 251 is preferably directed to a quench unit 252 wherein the light olefins-containing stream 251 contacts a quench medium under conditions effective to condense at least a portion of the readily condensable components contained in light olefins-containing stream 251. Specifically, in quench unit 252, the light olefin-containing stream 251 is cooled and water and other readily condensable components are condensed. The condensed components, which comprise a substantial amount of water, are withdrawn from the quench unit 252 through a quench bottoms stream 253. A portion of the condensed components are circulated through pumparound stream 255 back to the top of quench unit 252. The components in pumparound stream 255 preferably are cooled in one or more heat exchangers 256 prior to being reintroduced into quench unit 252. As shown, pumparound stream 255, is cooled in heat exchanger 256 to form cooled pumparound stream 257, e.g., the quench medium to be reintroduced into quench unit 252. In this manner, cooled pump around stream 257 cools light olefin-containing stream 251 in quench unit 252 under conditions effective to condense out the readily condensable components contained therein. Olefin-containing vapor is yielded from the quench unit 252 via quench overhead stream 254, which preferably is directed to one or more, preferably a plurality of compression stages.

For purposes of clarity, a single compression stage 258 is illustrated in FIG. 2, although the compression system optionally includes two, three, four or more compression stages. Compression stage 258 comprises a compressor 259, a heat exchanger 261, and a knockout drum 263. In operation, quench overhead stream 254 is introduced into compressor 259 which compresses quench overhead stream 254 and forms compressed stream 260. Compressed stream 260 is directed to a heat exchanger 261 wherein the compressed stream 260 indirectly contacts a cooling medium under conditions effective to form cooled compressed stream 262. Cooled compressed stream 262 is then directed to a knockout drum 263. In knockout drum 263, readily condensable components contained in cooled compressed stream 262 are allowed to condense from non-readily condensable components contained therein. Thus, knockout drum 263 forms a condensate stream 265 and a vapor stream 264. Optionally, if cooled compressed stream 262 contains readily condensable aqueous and hydrocarbon components, then knockout drum 263 optionally is a two or three-phase separation unit adapted to form an aqueous condensate stream and a hydrocarbon condensate stream.

Vapor stream 264 ideally is directed to a caustic wash unit 276 for removal of entrained acid gases such as CO2. Thus, as with caustic wash unit 133, illustrated in FIG. 1, vapor stream 264 contacts a caustic medium introduced via caustic stream 269 under conditions effective to form CO2 depleted stream 266 and spent caustic stream 267. Caustic wash unit 276 preferably comprises one or more pumparound streams 268 that withdraw at least partially spent caustic medium from the caustic wash unit 276 and reintroduces the at least partially spent caustic into the caustic wash unit 276. Optionally, caustic wash unit 276 includes a water wash feature. In this embodiment, gaseous components contained in the top region of caustic wash unit 276 may contain a minor amount of entrained caustic medium. The water wash feature of the caustic wash unit 276 provides for the ability to remove at least a portion, preferably a majority, of the entrained caustic medium contained in caustic wash unit 276.

CO2 depleted stream 266 is then preferably directed to a drying unit 271. As described above with reference to FIG. 1, CO2 depleted stream 266 ideally contacts a drying medium in drying unit 271 under conditions effective to remove water therefrom and form dry stream 272. Dry stream 272 is then directed to a separation system 273, which is adapted to separate the components contained in dry stream 272. As shown, separation system 273 separates dry stream 272 into an ethylene-containing stream 274 and a propylene-containing stream 275. More likely, however, separation system 273 yields more than two derivative streams, as described in greater detail below. Two preferred separation systems are disclosed in greater detail in co-pending U.S. patent application Ser. No 10/383,204, filed Mar. 6, 2003, and in co-pending U.S. patent application Ser. No. ______, the entireties of which are incorporated herein by reference.

Integrated Steam Cracking and Oxygenate to Olefin Reaction System

The present invention provides an integrated steam cracking and oxygenate to olefin reaction system. By integrating a steam cracking system with an OTO reaction system, several benefits may be achieved.

In one aspect of the present invention, equipment count is reduced over having separate steam cracking and OTO reaction systems. Specifically, an integrated system according to the present invention may share one or more of the following units: quench units (direct or indirect), compression systems, caustic wash units, water wash units, drying units, separation systems and/or utility and product storage systems. As a result, start up costs can be significantly reduced over providing separate independent steam cracking and OTO reaction systems for the production of light olefins. For example, if it is desired to increase light olefin production by building an OTO reactor, the OTO reactor can be integrated into existing steam cracking processing equipment. By so doing, the start up costs for getting an OTO reactor operational are significantly reduced over building an entire OTO reaction system, which would include an independent quench unit, compression system, caustic wash unit and separation train, in addition to the OTO reactor.

Additionally, should the steam cracking unit or the OTO reaction system require repairs or otherwise require shutdown, the decreased production resulting from the down time of that unit can be minimized by increasing the production of the other functional unit. For example, if the steam cracking unit is shut down for repairs, the production of the OTO reaction unit optionally can be increased in order to make up for the lost production of the steam cracking unit. In this manner, a consistent light olefin production can be advantageously maintained over extended periods of time.

Another significant advantage of an integrated system is that compression efficiency per pound of light olefins produced can be significantly increased over conventional steam cracking compression systems. A conventional steam cracking compression system has an efficiency of from about 35 (26) to about 55 (41) horsepower (KW) per metric kiloton per annum of light olefins produced. An OTO reaction system, in contrast, can have an efficiency of from about 10 (7.4) to about 14 (10) horsepower (KW) per metric kiloton per annum of light olefins produced. This difference in compression power requirements is due, in large part, to the greater molar ratio of light olefins to other components produced in an OTO effluent stream over steam cracking systems.

If the effluent stream from a steam cracking system is combined with the effluent stream from an OTO reaction system prior to effluent compression, then the compressor system efficiency in the integrated system can be increased over compression systems found in conventional steam cracking systems, thereby resulting in a significant commercial savings. Specifically, the power in an integrated compression system optionally ranges from about 10 to about 50, from about 15 to about 35, or from about 15 to about 25 horsepower per metric kiloton per annum of light olefins produced. Maximizing compressor efficiency is particularly important for centrifugal compressors, which are preferred, as centrifugal compressors typically have a fixed maximum power capacity. The process of increasing the efficiency of existing compressor systems by increasing the molar ratio of light olefins to other components over existing steam cracking compression systems is referred to herein as “debottlenecking.”

Optionally, an effluent stream from a steam cracking system is combined with a partially compressed stream from OTO reaction system. Alternatively, an effluent stream from an OTO reaction system is combined with a partially compressed stream from a steam cracking system. In these embodiments, the effluent streams optionally are combined between compression stages or in the middle of a compression stage, thus providing a partially integrated compression system. This embodiment may be desirable if, prior to compression, the streams have differing pressures. Preferably, at the point where the streams are combined, the pressure of the OTO effluent stream does not differ from the pressure of the steam cracking effluent stream by more than 10 psia (68.9 kpaa), by no more than 5 psia (34.5 kpaa), or no more than 1 psia (6.9 kPaa). For example, if one effluent stream has a greater pressure than the other effluent stream, then the less compressed stream optionally is compressed in one or more initial stages, and is then combined with the other effluent stream. In this embodiment, the debottlenecking potential of the present invention may be partially, but not fully, realized. Specifically, depending on the pressure of the effluent streams when they are combined, the power of the partially integrated compression system optionally ranges from about 15 (11.2) to about 55 (41), from about 20 (15) to about 40 (30), or from about 20 (15) to about 30 (22) horsepower (kwatt) per metric kiloton per annum of light olefins produced.

In a gas to olefin and OTO reaction systems, substantially less NOx and CO2 is produced per pound of light olefins than in steam cracking systems. Methanol is made from natural gas via the production of syngas as an intermediate step. Syngas production is an energy intensive process due to the endothermic nature of methane reforming. Although the process of synthesizing methanol from syngas is exothermic, the overall methanol synthesis process has high energy requirements because of the syngas production process. On the other hand, the OTO reaction process is an exothermic reaction, which is very energy efficient. The OTO reaction process, excluding methanol production, emits about 70% less NOx and about 60% less CO2 per unit of ethylene and propylene than steam cracking. Table I, below, compares energy usage and pollution production of OTO reaction systems, Ethane Steam Cracking Systems and Naphtha Steam Cracking Systems.

TABLE I
Comparing Energy and Pollution Production in Ethane Steam Cracking Systems,
Naphtha Steam Cracking Systems and OTO Reaction Systems

| | $\frac{\begin{matrix}Energy \\ Usage *\end{matrix}}{{C2}^{=}}$ BTU/lb (kJ/kg) | $\frac{( {C2}^{=} + {C3}^{=} )}{{C2}^{=}}$ lb/lb (kg/kg) | $\frac{\begin{matrix}Energy \\ Usage *\end{matrix}}{( {C2}^{=} + {C3}^{=} )}$ BTU/lb (kJ/kg) | $\frac{{CO}_{2}}{{C2}^{=}}$ lb/lb (kg/kg) | $\frac{{NO}_{x}}{\begin{matrix}{C2}^{=} \\ ( \times {10}^{- 4} )\end{matrix}}$ lb/lb (kg/kg) | $\frac{{CO}_{2}}{( {C2}^{=} + {C3}^{=} )}$ lb/lb (kg/kg) | $\frac{{NO}_{x}}{\begin{matrix}( {C2}^{=} + {C3}^{=} ) \\ ( \times {10}^{- 4} )\end{matrix}}$ lb/lb (kg/kg) |
|---|---|---|---|---|---|---|---|
| Ethane to | | | | | | | |
| Olefins | | | | | | | |
| LNG to | 814 | 1 | 814 | 0.10 | 0.41 | 0.10 | 0.41 |
| Ethane | (1,891) | | (1,891) | | | | |
| Ethane | 7,297 | 1 | 7,297 | 0.94 | 3.65 | 0.94 | 3.65 |
| Cracking | (16,956) | | (16,956) | | | | |
| Total | 8,111 | | 8,111 | 1.04 | 4.06 | 1.04 | 4.06 |
| | (18,847) | | (18,847) | | | | |
| Naphtha to | | | | | | | |
| Olefins | | | | | | | |
| Gas | 1,721 | 1.47 | 1,171 | 0.22 | 0.86 | 0.15 | 0.58 |
| Condensate | (3,999) | | (2,721) | | | | |
| Crude to | 8,107 | 1.47 | 5,515 | 1.04 | 4.05 | 0.71 | 2.76 |
| Naphtha | (18,838) | | (12,815) | | | | |
| Naphtha | 10,330 | 1.47 | 7,027 | 1.33 | 5.17 | 0.91 | 3.51 |
| Cracking | (24,003) | | (16,328) | | | | |
| Total | 18,437 | | 12,542 | 2.37 | 9.22 | 1.62 | 6.27 |
| (Crude to | (42,841) | | (29,143) | | | | |
| Naphtha) | | | | | | | |
| Gas to | | | | | | | |
| Olefins | | | | | | | |
| Gas to | 8,398 | 2 | 4,199 | 1.08 | 4.20 | 0.54 | 2.10 |
| Methanol | (19,514) | | (9,757) | | | | |
| Methanol to | 3,842 | 2 | 1,921 | 0.65 | 1.92 | 0.32 | 0.96 |
| Olefins | (8,927) | | (4,464) | | | | |
| Total | 12,240 | | 6,120 | 1.73 | 6.12 | 0.86 | 3.06 |
| | (28,441) | | (14,221) | | | | |

*These figures are based on the lower heating value.

The decreased pollution production in gas to olefins and OTO reaction systems provides a significant advantage for an integrated system according to the present invention as the amount of pollutants produced per pound of light olefins produced can be significantly lower in the integrated system than in conventional steam cracking systems. Thus, an additional benefit of integrating a steam cracking system with an OTO reaction system is that the amount of pollutants produced per pound of light olefins produced in an integrated system will be significantly less than the amount of pollutants produced per pound of light olefins produced in a steam cracking system. Conventional steam cracking systems produce a significant amount of pollutants such as NOx, and greenhouse gasses such as CO2. In contrast, OTO reaction systems are much cleaner than steam cracking systems, potentially making about 14 to about 33 weight percent less NOx and greenhouse gases (e.g., CO2) than steam cracking systems. Specifically, conventional steam cracking produces carbon dioxide at a rate of about 1.04 to about 1.62 gram CO2 per gram of ethylene and propylene produced. An OTO reaction system, in contrast, will have a carbon dioxide production of from about 0.7 to about 0.9 grams CO2 per gram of light olefins produced.

Additionally, in an integrated system, the amount of NOx emissions produced per amount of light olefins produced will be significantly less than in conventional steam cracking systems. Conventional steam cracking produces nitrous oxide from about 4.06×10−4 to about 6.27×10−4 grams NOx per gram of ethylene and propylene produced. An OTO reaction system, in contrast, can have a nitrous oxide production of from about 3.0×10−4 to about 3.5×10−4 grams NOx per gram of light olefins produced.

In another embodiment of the present invention, the volume ratio of the effluent received from a steam cracking system to effluent received from an OTO reaction system in the integrated system can be varied, as necessary, to provide desirable light olefins production, while ensuring compliance with applicable pollution standards. For example, in one embodiment of the present invention, the pollutants produced in an integrated system are monitored, and the volume ratio of effluent received in the integrated system from the steam cracking unit to effluent received from the OTO reaction system is varied responsive to a determination in the monitoring step that the pollutants have exceeded or fallen below a predetermined threshold value.

The invention, as discussed below, is generally described with regard to integrating an OTO reaction system into an existing steam cracking system in order to reduce the amount of pollutants produced in steam cracking per pound of light olefins produced and in order to take advantage of the debottlenecking characteristics of the integrated system. The reverse, however, is also possible. That is, it is within the scope of the present invention that a steam cracking system can be integrated into an existing OTO reaction system. Whether the original components were originally designed and produced for an OTO reaction system or for a steam cracking system is of little import so long as the units shared are capable of processing a combined stream.

In the integrated system, an effluent stream from the steam cracking system is combined, at some point, with an effluent stream from an OTO reaction system. The point at which these streams are to be combined may vary widely depending on available equipment, pollution concerns, and/or compression system loads.

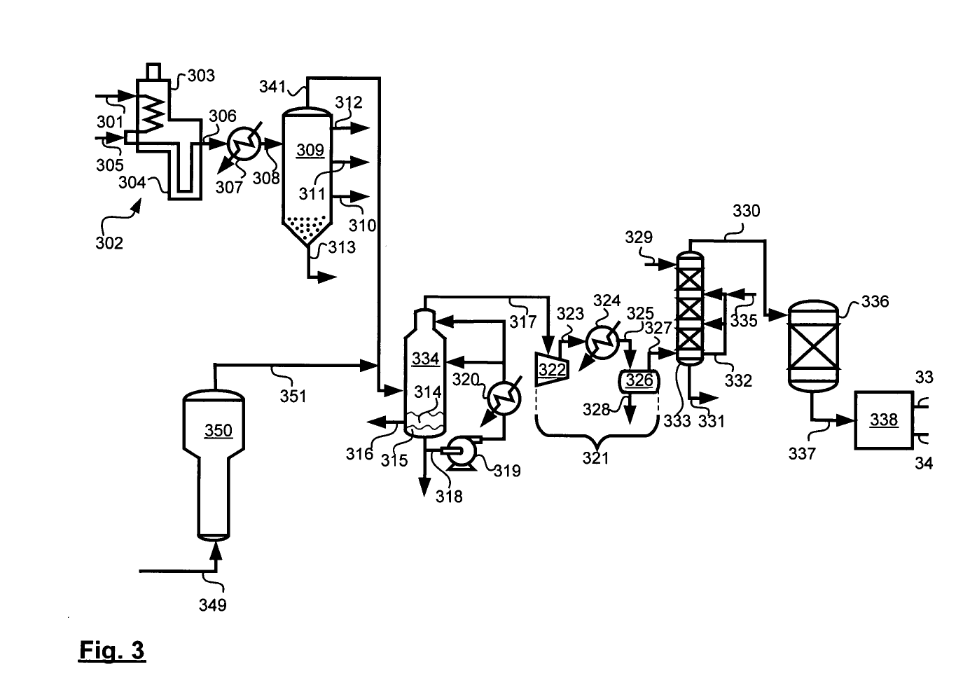

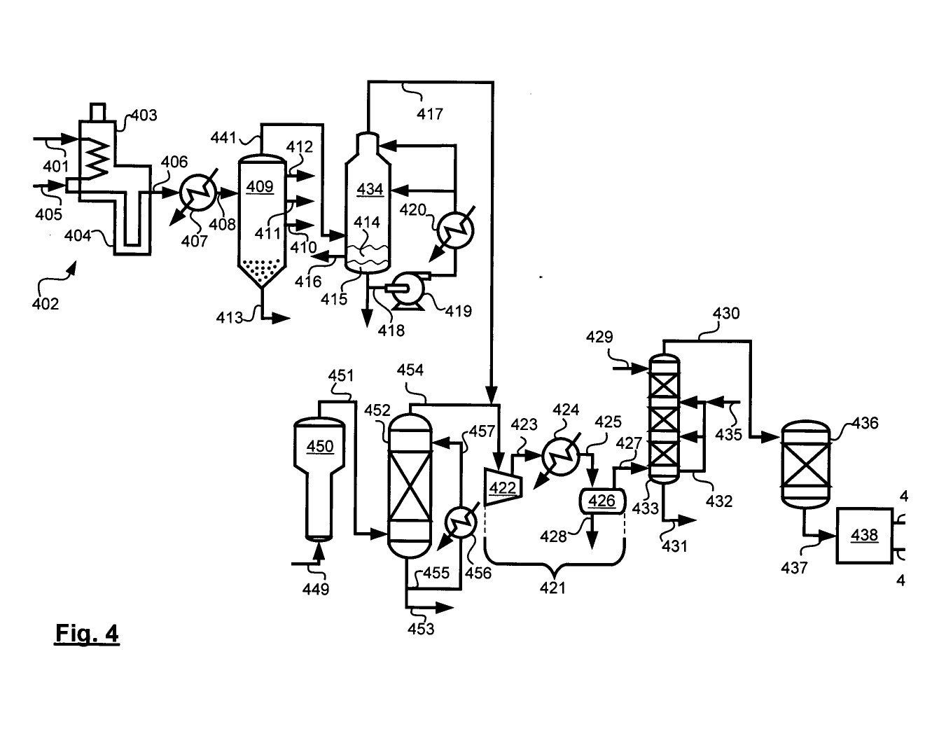

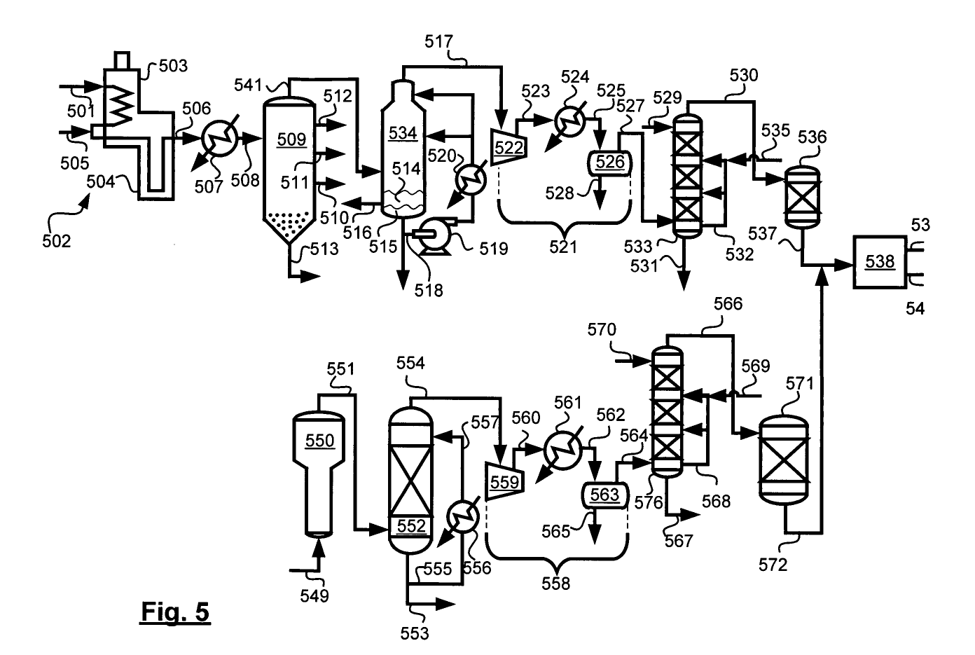

FIGS. 3-5, discussed in detail below, illustrate three possible integrated systems. In the first system, shown in FIG. 3, an effluent stream from a steam cracking system is combined with an effluent stream from an OTO reaction system just before direct effluent quenching. In FIG. 4, an effluent stream from a steam cracking system is combined with an effluent stream from an OTO reaction system after direct product quenching, but before effluent compression. In FIG. 5, an effluent stream from a steam cracking system is combined with an effluent stream from an OTO reaction system after effluent compression, but before entering a separation system, which is adapted to separate one or more of the components in the combined compressed effluent stream. Each of these systems will now be discussed in turn.

A. Pre-Quench Integration

FIG. 3 illustrates one embodiment of an integrated system, wherein a reaction effluent from an OTO reaction system is combined with an effluent from a steam cracking system prior to direct product quenching. This embodiment minimizes equipment count in the integrated system and provides a commensurate savings in start up costs.

As shown, a C2 to C40 containing feedstock 301 is directed to steam cracking unit 302. Preferably, feedstock 301 comprises C2 to C30 hydrocarbons, and more preferably C2 to C20 hydrocarbons. Steam cracking unit 302 preferably comprises a preheat region 303 and a cracking region 304. In the preheat region 303, the feedstock 301 is heated to form a heated feedstock stream having a temperature of from about 200° F. (93° C.) to about 450° F. (232° C.). Steam from steam containing line 305 preferably is admixed with the heated feedstock 301, which is then directed to cracking region 304. In cracking region 304, the steam containing feedstock 301 is further heated under conditions effective to “crack” the hydrocarbons and produce an effluent stream 306 comprising fuel oil, gas oil, pyrolysis gasoline, and C5− hydrocarbon components (including ethylene and propylene).

Effluent stream 306 optionally is directed to indirect quench unit 307 for indirect effluent stream quenching. In the indirect quench unit 307, effluent stream 306 is cooled under conditions effective to form cooled stream 308. Cooled stream 308 is then preferably directed to one or more fractionation units 309. In fractionation unit 309, cooled stream 308 is subjected to temperature and pressure conditions that are effective to separate the cooled stream 308 into a plurality of derivative non-aliquot streams. As shown, cooled stream 308 is fractionated in fractionation unit 309 to form fuel oil stream 310, gas oil stream 311, gasoline stream 312, and light hydrocarbon stream 341 (containing C5− hydrocarbons including ethylene and propylene). In one integrated embodiment of the present invention, light hydrocarbon stream 341 is combined with a reaction effluent stream from an OTO reactor, as described in greater detail below.

Carbonaceous deposits (coke) are formed as a byproduct of the steam cracking process. These carbonaceous deposits exit the steam cracking unit 302 via effluent stream 306 and are directed through indirect quench unit 307 and cooled stream 308 to fractionation unit 309. Upon introduction into fractionation unit 309, the carbonaceous deposits settle and are separated from the liquid components contained in fractionation unit 309. The settled carbonaceous deposits in fractionation unit 309 ideally are withdrawn therefrom, as shown by coke removal stream 313.

FIG. 3 also illustrates an OTO reaction system. As shown, an oxygenate containing feedstock 349 is directed to an OTO reaction unit 350, wherein the oxygenate, e.g., methanol, in oxygenate-containing feedstock 349 contacts a molecular sieve catalyst composition under conditions effective to convert at least a portion of the oxygenate in the oxygenate-containing feedstock 349 to light olefins. The light olefins formed in reaction unit 350 are yielded therefrom in light olefins-containing stream 351.

At least a portion of light olefins-containing stream 351 preferably is combined with light hydrocarbon stream 341 from the steam cracking system to form a combined stream, which ideally is directed to a quench unit 334, wherein readily condensable components in the combined stream are condensed from non-readily condensable components contained therein. In another embodiment, not shown, the effluent streams are combined inside the quench unit 334. That is, the combining step optionally occurs in the quench unit 334, or external to the quench unit 334. In an alternative embodiment, the light olefins-containing stream 351 is directed to a standalone quench unit, not shown, to capture excess water prior to introduction into the integrated quench unit 334.

Specifically, in quench unit 334, the combined stream or a portion thereof contacts a quenching medium under conditions effective to condense out the readily condensable components contained therein. The readily condensable components in light hydrocarbon stream 341 comprise aqueous and hydrocarbon components, which separate in the quench unit 334 and form two condensed phases, specifically, hydrocarbon phase 314 and aqueous phase 315.

As shown, hydrocarbon phase 314 contains mostly C6 hydrocarbon components, which are withdrawn from quench unit 334 via pygas stream 316. Aqueous phase 315 is withdrawn from quench unit 334 via condensate stream 318 with the assistance of pump 319. Pump 319 directs a portion of condensate stream 318 through heat exchanger 320, which cools condensate stream 318. The cooled condensate stream 318 is then directed back to quench unit 334 wherein the cooled condensate stream 318 serves as a quench medium to condense out readily condensable components that enter quench unit 334 via light hydrocarbon stream 341. Optionally, cooled condensate stream 318 is separated into a plurality of streams, which introduce the cooled condensate stream 318 into quench unit 334 through a plurality of inlets in quench unit 334. Non-readily condensable components contained in light hydrocarbon stream 341 exit quench unit 334 via quench overhead stream 317.

In this embodiment, the aqueous phase 315 from the integrated quench unit may contain a significant amount of residual oxygenate components, such as methanol, ethanol, dimethyl ether, ethanal, propanal, acetone, isopropyl alcohol or mixtures thereof, which optionally is separated from the integrated quench bottoms stream and recycled to the OTO reaction unit for conversion to light olefins. In this embodiment, all or a portion of the quench bottoms stream from the integrated quench unit is directed to an oxygenate separation unit (not shown), e.g., an oxygenate fractionation unit, designed to separate oxygenate components such as methanol, ethanol and DME from water. In the oxygenate separation unit, the aqueous quench bottoms stream (aqueous phase 315), or a portion thereof, is subjected to conditions, e.g., temperature and pressure, effective to separate the quench bottoms stream into an oxygenate-containing overhead stream and a water-containing bottoms stream, wherein the oxygenate-containing overhead stream contains at least a majority of the oxygenated components contained in the quench bottoms stream, and the water-containing bottoms stream contains at least a majority of the water from the quench bottoms stream. All or a portion of the oxygenate-containing overhead stream is directed to the OTO reaction unit for conversion to light olefins. Optionally, one or more of the following streams, discussed in detail below, are also directed to the oxygenate separation unit for removal of residual oxygenate compounds contained therein: condensed components from an integrated or non-integrated compression system (e.g, from the knock out drums thereof); the bottoms stream from an integrated or non-integrated oxygenate (e.g., methanol) wash unit; and/or the bottoms stream from an integrated or non-integrated water wash unit.

Quench overhead stream 317 ideally is then compressed in one or more compression stages, preferably a plurality of compression stages. FIG. 3 illustrates a single compression stage 321, which comprises a compressor 322, a heat exchanger 324 and a knockout drum 326. However, the compression system optionally comprises two, three, four or more compression stages. Reverting to FIG. 3, quench overhead stream 317 is directed to compressor 322, which compresses quench overhead stream 317 and forms compressed stream 323. Compressed stream 323 is then directed to a heat exchanger 324 in which the compressed stream 323 indirectly contacts a cooling medium under conditions effective to cool compressed stream 323 and form cooled compressed stream 325. Cooled compressed stream 325 is then directed to a knockout drum 326, wherein readily condensable components are allowed to condense from non-readily condensable components. The readily condensable components exit knockout drum 326 via condensed stream 328. If the readily condensable components contained in cooled compressed stream 325 comprise aqueous and hydrocarbon components, then the knockout drum 326 optionally is a two- or three-phase knockout drum, not shown, which is adapted to form an aqueous condensed stream, a hydrocarbon condensed stream, and a vapor stream. As shown, knockout drum 326 is adapted to separate condensed components in condensed stream 328 from gaseous components, which exit knockout drum 326 via vapor stream 327.

Vapor stream 327 ideally is then directed to a caustic wash unit 333 for removal of entrained acid gases such as CO2. That is, caustic wash unit 333 removes CO2 and other entrained acid gases from the vapor stream 327, or a portion thereof, and forms a CO2 depleted stream 330. The vapor stream 327 preferably contains relatively few hydrocarbon components that cause fouling problems in such acid gas treatment systems.

Caustic wash unit 333 comprises one or more, preferably a plurality of beds (three are shown) and a pumparound stream 332. Pumparound stream 332 withdraws a portion of the spent caustic from caustic wash unit 333 and recirculates it to one or more higher portions on the caustic wash unit 333. As shown, pumparound stream 332 withdraws a portion of the spent caustic solution from the caustic wash unit 333 and recirculates it into caustic wash unit 333 through two inlets. Fresh caustic 335 optionally is introduced into the pumparound stream 332 or directly into caustic wash unit 333. Optionally, water stream 329 is also introduced into caustic wash unit 333, preferably adjacent the top end thereof, in order to ensure that CO2 depleted stream 330 does not contain entrained caustic medium, or contains a minimal amount of entrained caustic medium. In this manner, caustic wash unit 333 functions to remove CO2 and other entrained acid gas components as well as functions as a water wash unit. Thus, upon introduction into caustic wash unit 333, vapor stream 327 contacts a caustic medium 335 under conditions effective to remove CO2 and other entrained acid gas components thereof, and form CO2 depleted stream 330 and spent caustic stream 331. CO2 depleted stream 330 is then directed to a drying unit 336. Optionally, CO2 depleted stream 330 is compressed in one or more additional stages, not shown, between caustic wash unit 333 and drying unit 336.

As shown in FIG. 3, CO2 depleted stream 330 contacts a drying agent in drying unit 336 under conditions effective to form dry stream 337. Dry stream 337 is then directed to a separation system 338, in which the components of dry stream 337 ideally are separated into two or more non-aliquot product streams. As shown, separation system 338 separates dry stream 337 into ethylene-containing stream 339 and propylene-containing stream 340. It is more likely, however, that separation system 338 separates the dry stream 337 into more than two derivative non-aliquot streams. Separation system 338 preferably comprises one or more separation units, preferably a plurality thereof. The separation units optionally include one or more distillation columns, fractionation columns, splitters, separators, wash columns, absorption units, or other separation devices.

B. Post-Quench, Pre-Compression Integration

FIG. 4 illustrates another embodiment of an integrated system, wherein the quench overhead stream from a steam cracking system is combined, at least in part, with a quench overhead stream from an OTO reaction system prior to compression. This embodiment provides reduced equipment count in the integrated system and provides a commensurate savings in start up costs. Additionally, since the integration occurs upstream of the process gas compression system, this embodiment provides desirable debottlenecking characteristics.

As shown, a C2 to C40 containing feedstock 401 is directed to steam cracking unit 402. Preferably, feedstock 401 comprises C2 to C30 hydrocarbons, and more preferably C2 to C20 hydrocarbons. Steam cracking unit 402 preferably comprises a preheat region 403 and a cracking region 404. In the preheat region 403, the feedstock 401 is heated to form a heated feedstock stream having a temperature of from about 200° F. (93° C.) to about 450° F. (232° C.). Steam from steam containing line 405 preferably is admixed with the heated feedstock 401, which is then directed to cracking region 404. In cracking region 404, the steam containing feedstock 401 is further heated under conditions effective to “crack” the hydrocarbons and produce an effluent stream 406 comprising fuel oil, gas oil, pyrolysis gasoline, and C5− hydrocarbon components (including ethylene and propylene).

Effluent stream 406 optionally is directed to indirect quench unit 407 for indirect effluent stream quenching. In the indirect quench unit 407, effluent stream 406 is cooled under conditions effective to form cooled stream 408. Cooled stream 408 is then preferably directed to one or more fractionation units 409. In fractionation unit 409, cooled stream 408 is subjected to temperature and pressure conditions that are effective to separate the cooled stream 408 into a plurality of derivative non-aliquot streams. As shown, cooled stream 408 is fractionated in fractionation unit 409 to form fuel oil stream 410, gas oil stream 411, gasoline stream 412, and light hydrocarbon stream 441 (containing C5− hydrocarbons including ethylene and propylene).

Carbonaceous deposits (coke) are formed as a byproduct of the steam cracking process. These carbonaceous deposits exit the steam cracking unit 402 via effluent stream 406 and are directed through indirect quench unit 407 and cooled stream 408 to fractionation unit 409. Upon introduction into fractionation unit 409, the carbonaceous deposits settle and are separated from the liquid components contained in fractionation unit 409. The settled carbonaceous deposits in fractionation unit 409 ideally are withdrawn therefrom, as shown by coke removal stream 413.

Light hydrocarbon stream 441 preferably is directed to a quench unit 434, wherein readily condensable components in the light hydrocarbon stream 441 are condensed from non-readily condensable components contained therein. Specifically, in quench unit 434, the light hydrocarbon stream 441 or a portion thereof contacts a quenching medium under conditions effective to condense out the readily condensable components contained therein. The readily condensable components in light hydrocarbon stream 441 comprise aqueous and hydrocarbon components, which separate in the quench unit 434 and form two condensed phases, specifically, hydrocarbon phase 414 and aqueous phase 415.

As shown, hydrocarbon phase 414 contains mostly C6 hydrocarbon components, which are withdrawn from quench unit 434 via pygas stream 416. Aqueous phase 415 is withdrawn from quench unit 434 via condensate stream 418 with the assistance of pump 419. Pump 419 directs a portion of condensate stream 418 through heat exchanger 420, which cools condensate stream 418. The cooled condensate stream 418 is then directed back to quench unit 434 wherein the cooled condensate stream 418 serves as a quench medium to condense out readily condensable components that enter quench unit 434 via light hydrocarbon stream 441. Optionally, cooled condensate stream 418 is separated into a plurality of streams, which introduce the cooled condensate stream 418 into quench unit 434 through a plurality of inlets in quench unit 434. Non-readily condensable components contained in light hydrocarbon stream 441 exit quench unit 434 via quench overhead stream 417. In one integrated embodiment of the present invention, quench overhead stream 417 is combined with a quench overhead stream from an OTO reactor, as described in greater detail below.

FIG. 4 also illustrates an OTO reaction system. As shown, an oxygenate containing feedstock 449 is directed to an OTO reaction unit 450, wherein the oxygenate, e.g., methanol, in oxygenate-containing feedstock 449 contacts a molecular sieve catalyst composition under conditions effective to convert at least a portion of the oxygenate in the oxygenate-containing feedstock 449 to light olefins. The light olefins formed in reaction unit 450 are yielded therefrom in light olefins-containing stream 451.

Light olefins-containing stream 451 is preferably directed to a quench unit 452 wherein the light olefins-containing stream 451 contacts a quench medium under conditions effective to condense at least a portion of the readily condensable components contained in light olefins-containing stream 451. Specifically, in quench unit 452, the light olefin-containing stream 451 is cooled and water and other readily condensable components are condensed. The condensed components, which comprise a substantial amount of water, are withdrawn from the quench unit 452 through a quench bottoms stream 453. A portion of the condensed components are circulated through pumparound stream 455 back to the top of quench unit 452. The components in pumparound stream 455 preferably are cooled in one or more heat exchangers 456 prior to being reintroduced into quench unit 452. As shown, pumparound stream 455 is cooled in heat exchanger 456 to form cooled pumparound stream 457, e.g., the quench medium to be reintroduced into quench unit 452. In this manner, cooled pump around stream 457 cools light olefin-containing stream 451 in quench unit 452 under conditions effective to condense out the readily condensable components contained therein. Olefin-containing vapor is yielded from the quench unit 452 via quench overhead stream 454, which preferably is directed to one or more, preferably a plurality, of compression stages. In the integrated embodiment illustrated in FIG. 4, at least a portion of quench overhead stream 417 from the steam cracking system is combined with at least a portion of quench overhead stream 454 from the OTO reaction system to form a combined stream, which ideally is directed to a shared compression system.

Thus, the combined stream ideally is compressed in one or more compression stages, preferably a plurality of compression stages. For clarity, FIG. 4 illustrates a single compression stage 421, which comprises a compressor 422, a heat exchanger 424 and a knockout drum 426. However, the compression system optionally comprises two, three, four or more compression stages. Reverting to FIG. 4, quench overhead stream 417 is directed to compressor 422, which compresses quench overhead stream 417 and forms compressed stream 423. Compressed stream 423 is then directed to a heat exchanger 424 in which the compressed stream 423 indirectly contacts a cooling medium under conditions effective to cool compressed stream 423 and form cooled compressed stream 425. Cooled compressed stream 425 is then directed to a knockout drum 426, wherein readily condensable components are allowed to condense from non-readily condensable components. The readily condensable components exit knockout drum 426 via condensed stream 428. If the readily condensable components contained in cooled compressed stream 425 comprise aqueous and hydrocarbon components, then the knockout drum 426 optionally is a two- or three-phase knockout drum, not shown, which is adapted to form an aqueous condensed stream, a hydrocarbon condensed stream, and a vapor stream. As shown, knockout drum 426 is adapted to separate condensed components in condensed stream 428 from gaseous components, which exit knockout drum 426 via vapor stream 427.

Vapor stream 427 ideally is then directed to a caustic wash unit 433 for removal of entrained acid gases such as CO2. That is, caustic wash unit 433 removes CO2 and other entrained acid gases from the vapor stream 427, or a portion thereof, and forms a CO2 depleted stream 430. The vapor stream 427 preferably contains relatively few hydrocarbon components that cause fouling problems in such acid gas treatment systems.

Caustic wash unit 433 comprises one or more, preferably a plurality of beds (three are shown) and a pumparound stream 432. Pumparound stream 432 withdraws a portion of the spent caustic from caustic wash unit 433 and recirculates it to one or more higher portions on the caustic wash unit 433. As shown, pumparound stream 432 withdraws a portion of the spent caustic solution from the caustic wash unit 433 and recirculates it into caustic wash unit 433 through two inlets. Fresh caustic 435 optionally is introduced into the pumparound stream 432 or directly into caustic wash unit 433. Optionally, water stream 429 is also introduced into caustic wash unit 433, preferably adjacent the top end thereof, in order to ensure that CO2 depleted stream 430 does not contain entrained caustic medium, or contains a minimal amount of entrained caustic medium. In this manner, caustic wash unit 433 functions to remove CO2 and other entrained acid gas components as well as functions as a water wash unit. Thus, upon introduction into caustic wash unit 433, vapor stream 427 contacts a caustic medium 435 under conditions effective to remove CO2 and other entrained acid gas components thereof, and form CO2 depleted stream 430 and spent caustic stream 431. CO2 depleted stream 430 optionally is then directed to a drying unit 436. Optionally, CO2 depleted stream 430 is compressed in one or more additional stages, not shown, between caustic wash unit 433 and drying unit 436.

As shown in FIG. 4, CO2 depleted stream 430 contacts a drying agent in drying unit 436 under conditions effective to form dry stream 437. Dry stream 437 is then directed to a separation system 438, in which the components of dry stream 437 ideally are separated into two or more non-aliquot product streams. As shown, separation system 438 separates dry stream 437 into ethylene-containing stream 439 and propylene-containing stream 440. It is more likely, however, that separation system 438 separates the dry stream 437 into more than two derivative non-aliquot streams. Separation system 438 preferably comprises one or more separation units, preferably a plurality thereof. The separation units optionally include one or more distillation columns, splitters, separators, wash columns, absorption units, or other separation devices.

C. Post-Compression Integration

FIG. 5 illustrates another embodiment of an integrated system, wherein a compressed stream from a steam cracking system is combined, at least in part, with a compressed stream from an OTO reaction system prior to separation of the individual components contained in the dried streams. In this embodiment, the compressed streams optionally share a caustic wash unit and/or a drying unit in addition to sharing a separation system. This embodiment provides reduced equipment count in the separation system of the integrated system and provides a commensurate savings in start up costs.

As shown, a C2 to C40 containing feedstock 501 is directed to steam cracking unit 502. Preferably, feedstock 501 comprises C2 to C30 hydrocarbons, and more preferably C2 to C20 hydrocarbons. Steam cracking unit 502 preferably comprises a preheat region 503 and a cracking region 504. In the preheat region 503, the feedstock 501 is heated to form a heated feedstock stream having a temperature of from about 200° F. (93° C.) to about 450° F. (232° C.). Steam from steam containing line 505 preferably is admixed with the heated feedstock 501, which is then directed to cracking region 504. In cracking region 504, the steam containing feedstock 501 is further heated under conditions effective to “crack” the hydrocarbons and produce an effluent stream 506 comprising fuel oil, gas oil, pyrolysis gasoline, and C5− hydrocarbon components (including ethylene and propylene).

Effluent stream 506 optionally is directed to indirect quench unit 507 for indirect effluent stream quenching. In the indirect quench unit 507, effluent stream 506 is cooled under conditions effective to form cooled stream 508. Cooled stream 508 is then preferably directed to one or more fractionation units 509. In fractionation unit 509, cooled stream 508 is subjected to temperature and pressure conditions that are effective to separate the cooled stream 508 into a plurality of derivative non-aliquot streams. As shown, cooled stream 508 is fractionated in fractionation unit 509 to form fuel oil stream 510, gas oil stream 511, gasoline stream 512, and light hydrocarbon stream 541 (containing C5− hydrocarbons including ethylene and propylene).

Carbonaceous deposits (coke) are formed as a byproduct of the steam cracking process. These carbonaceous deposits exit the steam cracking unit 502 via effluent stream 506 and are directed through indirect quench unit 507 and cooled stream 508 to fractionation unit 509. Upon introduction into fractionation unit 509, the carbonaceous deposits settle and are separated from the liquid components contained in fractionation unit 509. The settled carbonaceous deposits in fractionation unit 509 ideally are withdrawn therefrom, as shown by coke removal stream 513.

Light hydrocarbon stream 541 preferably is directed to a quench unit 534, wherein readily condensable components in the light hydrocarbon stream 541 are condensed from non-readily condensable components contained therein. Specifically, in quench unit 534, the light hydrocarbon stream 541 or a portion thereof contacts a quenching medium under conditions effective to condense out the readily condensable components contained therein. The readily condensable components in light hydrocarbon stream 541 comprise aqueous and hydrocarbon components, which separate in the quench unit 534 and form two condensed phases, hydrocarbon phase 514 and aqueous phase 515.

As shown, hydrocarbon phase 514 contains mostly C6 hydrocarbon components, which are withdrawn from quench unit 534 via pygas stream 516. Aqueous phase 515 is withdrawn from quench unit 534 via condensate stream 518 with the assistance of pump 519. Pump 519 directs a portion of condensate stream 518 through heat exchanger 520, which cools condensate stream 518. The cooled condensate stream 518 is then directed back to quench unit 534 wherein the cooled condensate stream 518 serves as a quench medium to condense out readily condensable components that enter quench unit 534 via light hydrocarbon stream 541. Optionally, cooled condensate stream 518 is separated into a plurality of streams, which introduce the cooled condensate stream 518 into quench unit 534 through a plurality of inlets in quench unit 534. Non-readily condensable components contained in light hydrocarbon stream 541 exit quench unit 534 via quench overhead stream 517.

Quench overhead stream 517 ideally is compressed in one or more compression stages, preferably a plurality of compression stages. For clarity, FIG. 5 illustrates a single compression stage 521, which comprises a compressor 522, a heat exchanger 524 and a knockout drum 526. However, the compression system optionally comprises two, three, four or more compression stages. Reverting to FIG. 5, quench overhead stream 517 is directed to compressor 522, which compresses quench overhead stream 517 and forms compressed stream 523. Compressed stream 523 is then directed to a heat exchanger 524 in which the compressed stream 523 indirectly contacts a cooling medium under conditions effective to cool compressed stream 523 and form cooled compressed stream 525. Cooled compressed stream 525 is then directed to a knockout drum 526, wherein readily condensable components are allowed to condense from non-readily condensable components. The readily condensable components exit knockout drum 526 via condensed stream 528. If the readily condensable components contained in cooled compressed stream 525 comprise aqueous and hydrocarbon components, then the knockout drum 526 optionally is a two- or three-phase knockout drum, not shown, which is adapted to form an aqueous condensed stream, a hydrocarbon condensed stream, and a vapor stream. As shown, knockout drum 526 is adapted to separate condensed components in condensed stream 528 from gaseous components, which exit knockout drum 526 via vapor stream 527.

Vapor stream 527 ideally is then directed to a caustic wash unit 533 for removal of entrained acid gases such as CO2. That is, caustic wash unit 533 removes CO2 and other entrained acid gases from the vapor stream 527, or a portion thereof, and forms a CO2 depleted stream 530. The vapor stream 527 preferably contains relatively few hydrocarbon components that cause fouling problems in such acid gas treatment systems.

Caustic wash unit 533 comprises one or more, preferably a plurality of beds (three are shown) and a pumparound stream 532. Pumparound stream 532 withdraws a portion of the spent caustic from caustic wash unit 533 and recirculates it to one or more higher portions on the caustic wash unit 533. As shown, pumparound stream 532 withdraws a portion of the spent caustic solution from the caustic wash unit 533 and recirculates it into caustic wash unit 533 through two inlets. Fresh caustic 535 optionally is introduced into the pumparound stream 532 or directly into caustic wash unit 533. Optionally, water stream 529 is also introduced into caustic wash unit 533, preferably adjacent the top end thereof, in order to ensure that CO2 depleted stream 530 does not contain entrained caustic medium, or contains a minimal amount of entrained caustic medium. In this manner, caustic wash unit 533 functions to remove CO2 and other entrained acid gas components as well as functions as a water wash unit. Thus, upon introduction into caustic wash unit 533, vapor stream 527 contacts a caustic medium 535 under conditions effective to remove CO2 and other entrained acid gas components thereof, and form CO2 depleted stream 530 and spent caustic stream 531. CO2 depleted stream 530 optionally is then directed to a drying unit 536. Optionally, CO2 depleted stream 530 is compressed in one or more additional stages, not shown, between caustic wash unit 533 and drying unit 536.

In this embodiment, a solid or liquid drying system can be used to remove water and/or additional oxygenated hydrocarbons from the effluent stream that is directed thereto, e.g., CO2 depleted stream 530. As shown in FIG. 5, CO2 depleted stream 530 contacts a drying agent in drying unit 536 under conditions effective to form dry stream 537. In one integrated embodiment of the present invention, dry stream 537 is combined with a dry stream 572 from an OTO reaction system prior to separation of the individual components contained in the dry streams, as described in greater detail below.

FIG. 5 also illustrates an OTO reaction system. As shown, an oxygenate containing feedstock 549 is directed to an OTO reaction unit 550, wherein the oxygenate, e.g., methanol, in oxygenate-containing feedstock 549 contacts a molecular sieve catalyst composition under conditions effective to convert at least a portion of the oxygenate in the oxygenate-containing feedstock 549 to light olefins. The light olefins formed in reaction unit 550 are yielded therefrom in light olefins-containing stream 551.

Light olefins-containing stream 551 is preferably directed to a quench unit 552 wherein the light olefins-containing stream 551 contacts a quench medium under conditions effective to condense at least a portion of the readily condensable components contained in light olefins-containing stream 551. Specifically, in quench unit 552, the light olefin-containing stream 551 is cooled and water and other readily condensable components are condensed. The condensed components, which comprise a substantial amount of water, are withdrawn from the quench unit 552 through a quench bottoms stream 553. A portion of the condensed components are circulated through pumparound stream 555 back to the top of quench unit 552. The components in pumparound stream 555 preferably are cooled in one or more heat exchangers 556 prior to being reintroduced into quench unit 552. As shown, pumparound stream 555 is cooled in heat exchanger 556 to form cooled pumparound stream 557, e.g., the quench medium to be reintroduced into quench unit 552. In this manner, cooled pump around stream 557 cools light olefin-containing stream 551 in quench unit 552 under conditions effective to condense out the readily condensable components contained therein. Olefin-containing vapor is yielded from the quench unit 552 via quench overhead stream 554, which preferably is directed to one or more, preferably a plurality, of compression stages.

For purposes of clarity, a single compression stage 558 is illustrated in FIG. 5, although the compression system optionally includes two, three, four or more compression stages. Compression stage 558 comprises a compressor 559, a heat exchanger 561, and a knockout drum 563. In operation, quench overhead stream 554 is introduced into compressor 559 which compresses quench overhead stream 554 and forms compressed stream 560. Compressed stream 560 is directed to a heat exchanger 561 wherein the compressed stream 560 indirectly contacts a cooling medium under conditions effective to form cooled compressed stream 562. Cooled compressed stream 562 is then directed to a knockout drum 563. In knockout drum 563, readily condensable components contained in cooled compressed stream 562 are allowed to condense from non-readily condensable components contained therein. Thus, knockout drum 563 forms a condensate stream 565 and a vapor stream 564. Optionally, if cooled compressed stream 562 contains readily condensable aqueous and hydrocarbon components, then knockout drum 563 optionally is a two- or three-phase separation unit adapted to form an aqueous condensate stream and a hydrocarbon condensate stream.

Vapor stream 564 ideally is directed to a caustic wash unit 576 for removal of entrained acid gases such as CO2. Thus, as with caustic wash unit 133, illustrated in FIG. 1, vapor stream 564 contacts a caustic medium introduced via caustic stream 569 under conditions effective to form CO2 depleted stream 566 and spent caustic stream 567. Caustic wash unit 576 preferably comprises one or more pumparound streams 568 that withdraw at least partially spent caustic medium from the caustic wash unit 576 and reintroduces the at least partially spent caustic into the caustic wash unit 576. Optionally, caustic wash unit 576 includes a water wash feature. In this embodiment, gaseous components contained in the top region of caustic wash unit 576 may contain a minor amount of entrained caustic medium. The water wash feature of the caustic wash unit 576 provides for the ability to remove at least a portion, preferably a majority, of the entrained caustic medium contained in caustic wash unit 576.

CO2 depleted stream 566 is then preferably directed to a drying unit 571. As described above with reference to FIG. 1, CO2 depleted stream 566 ideally contacts a drying medium in drying unit 571 under conditions effective to remove water therefrom and form dry stream 572. In the integrated embodiment illustrated in FIG. 5, at least a portion of dry stream 572 from the OTO reaction system is combined with at least a portion of dry stream 537 from the steam cracking system to form a combined stream, which ideally is directed to a shared separation system. Thus, the combined stream is directed to a separation system 538, in which the components of dry stream 537 ideally are separated into two or more non-aliquot product streams. As shown, separation system 538 separates dry stream 537 into ethylene-containing stream 539 and propylene-containing stream 540. It is more likely, however, that separation system 538 separates the dry stream 537 into more than two derivative non-aliquot streams. Separation system 538 preferably comprises one or more separation units, preferably a plurality thereof. The separation units optionally include one or more distillation columns, splitters, separators, wash columns, absorption units, or other separation devices.

Ethylene and Propylene Disposition

The ethylene and propylene streams formed according to this invention can be polymerized to form plastic compositions, e.g., polyolefins, particularly polyethylene and polypropylene. Any conventional process for forming polyethylene or polypropylene can be used. Catalytic processes are preferred. Particularly preferred are metallocene, Ziegler/Natta, aluminum oxide and acid catalytic systems. See, for example, U.S. Pat. Nos. 3,258,455; 3,305,538; 3,364,190; 5,892,079; 4,659,685; 4,076,698; 3,645,992; 4,302,565; and 4,243,691, the catalyst and process descriptions of each being expressly incorporated herein by reference. In general, these methods involve contacting the ethylene or propylene product with a polyolefin-forming catalyst at a pressure and temperature effective to form the polyolefin product.

In one embodiment of this invention, the ethylene or propylene product is contacted with a metallocene catalyst to form a polyolefin. Desirably, the polyolefin forming process is carried out at a temperature ranging between about 50° C. and about 320° C. The reaction can be carried out at low, medium or high pressure, being anywhere within the range of about 1 bar to about 3200 bar. For processes carried out in solution, an inert diluent can be used. In this type of operation, it is desirable that the pressure be at a range of from about 10 bar to about 150 bar, and preferably at a temperature range of from about 120° C. to about 250° C. For gas phase processes, it is preferred that the temperature generally be within a range of about 60° C. to 120° C., and that the operating pressure be from about 5 bar to about 50 bar.

In addition to polyolefins, numerous other olefin derivatives can be formed from the ethylene, propylene and C4+ olefins, particularly butylene, separated according to this invention. The olefins separated according to this invention can also be used in the manufacture of such compounds as aldehydes, acids such as C2-C13 mono carboxylic acids, alcohols such as C2-C12 mono alcohol, esters made from the C2-C12 mono carboxylic acids and the C2-C12 mono alcohols, linear alpha olefins, vinyl acetate, ethylene dicholoride and vinyl chloride, ethylbenzene, ethylene oxide, cumene, acrolein, allyl chloride, propylene oxide, acrylic acid, ethylene-propylene rubbers, and acrylonitrile, and trimers and dimers of ethylene and propylene. The C4+ olefins, butylene in particular, are particularly suited for the manufacture of aldehydes, acids, alcohols, esters made from C5-C13 mono carboxylic acids and C5-C13 mono alcohols and linear alpha olefins.

Having now fully described the invention, it will be appreciated by those skilled in the art that the invention may be performed within a wide range of parameters within what is claimed, without departing from the spirit and scope of the invention.

1. A process for forming light olefins, wherein the process comprises the steps of:
(a) contacting hydrocarbons with steam in a cracking furnace under first conditions effective to form a first effluent stream containing fuel oil, gas oil, gasoline and light olefins;
(b) contacting methanol with a molecular sieve catalyst in a methanol-to-olefin reactor under second conditions effective to form a second effluent stream containing light olefins, C4+ hydrocarbons and water;
(c) combining at least a portion of the first effluent stream and at least a portion of the second effluent stream to form a combined stream;
(d) contacting the combined stream with a quench medium in a quench unit under conditions effective to form a quench overhead stream and a quench bottoms stream, wherein the quench overhead stream contains at least a majority of the light olefins that were present in the combined stream, and wherein the quench bottoms stream contains at least a portion of the water and condensed components; and
(e) fractionating at least a portion of the quench overhead stream to form a light olefins product stream and one or more C4+ hydrocarbon streams, wherein the light olefins product stream contains a majority of the light olefins that were present in the at least a portion of the quench overhead stream.

2. The process of claim 1, wherein the process further comprises the step of:
(f) compressing at least a portion of the quench overhead stream to form a compressed stream, wherein the at least a portion of the quench overhead stream that is fractionated in step (e) comprises at least a portion of the compressed stream.

3. The process of claim 2, wherein the compressing in step (f) has a load of from about 10 to about 50 horsepower per metric kiloton per annum light olefins produced.

4. The process of claim 3, wherein the load is from about 15 to about 25 horsepower per metric kiloton per annum light olefins produced.

5. The process of claim 1, wherein the fractionating in step (e) forms a fuel oil stream, a gas oil stream, a gasoline stream, and the light olefins-containing stream, wherein the fuel oil stream contains at least a majority of the fuel oil that was present in the first effluent stream, wherein the gas oil stream contains at least a majority of the gas oil that was present in the first effluent stream, and wherein the gasoline stream contains at least a majority of the gasoline that was present in the first effluent stream.

6. The process of claim 1, wherein the quench overhead stream contains carbon dioxide, the process further comprising the step of:
(f) contacting at least a portion of the quench overhead stream with a caustic medium in a caustic wash unit under conditions effective to remove at least a portion of the carbon dioxide from the quench overhead stream.

7. The process of claim 1, wherein the process further comprises the step of:
(f) heating the quench bottoms stream in an oxygenate fractionation unit under conditions effective to form an oxygenate-containing overhead stream and a water-containing bottoms stream, wherein the oxygenate-containing overhead stream contains at least a majority of oxygenated components that were present in the quench bottoms stream, and wherein the water-containing bottoms stream contains at least a majority of the water that was present in the quench bottoms stream.

8. The process of claim 7, wherein the process further comprises the step of:
(g) directing at least a portion of the oxygenate-containing overhead stream to the methanol-to-olefin reactor for conversion to light olefins.

9. The process of claim 1, wherein the process further comprises the steps of:
(f) cooling a portion of the quench bottoms stream to form a cooled stream; and
(g) introducing the cooled stream into the quench unit to serve as the quench medium.

10. The process of claim 1, wherein step (c) occurs in the quench unit.

11. The process of claim 1, wherein step (c) occurs external to the quench unit.

12. The process of claim 1, wherein the light olefins product stream contains at least 50 weight percent light olefins, based on the total weight of the light olefins product stream.

13. The process of claim 12, wherein the light olefins product stream contains at least 75 weight percent light olefins, based on the total weight of the light olefins product stream.

14. The process of claim 13, wherein the light olefins product stream contains at least 90 weight percent light olefins, based on the total weight of the light olefins product stream.

15. A process for forming light olefins, wherein the process comprises the steps of:
(a) contacting hydrocarbons with steam in a cracking furnace under first conditions effective to form a first effluent stream containing fuel oil, gas oil, gasoline and light olefins;
(b) separating the first effluent stream into a light olefins-containing stream and one or more heavy hydrocarbon streams, wherein the light olefins-containing stream contains at least a majority of the light olefins that were present in the first effluent stream;
(c) contacting methanol with a molecular sieve catalyst in a methanol-to-olefin reactor under second conditions effective to form a second effluent stream containing light olefins, C4+ hydrocarbons and water;
(d) combining at least a portion of the light olefins-containing stream and at least a portion of the second effluent stream to form a combined stream;
(e) contacting the combined stream with a quench medium in a quench unit under conditions effective to form a quench overhead stream and a quench bottoms stream, wherein the quench overhead stream contains at least a majority of the light olefins that were present in the combined stream, and wherein the quench bottoms stream contains at least a portion of the water and condensed components; and
(f) fractionating at least a portion of the quench overhead stream to form a light olefins product stream and one or more C4+ hydrocarbon streams, wherein the light olefins product stream contains a majority of the light olefins that were present in the at least a portion of the quench overhead stream.

16. The process of claim 15, wherein the process further comprises the step of:
(g) compressing at least a portion of the quench overhead stream to form a compressed stream, wherein the at least a portion of the quench overhead stream that is fractionated in step (f) comprises at least a portion of the compressed stream.

17. The process of claim 16, wherein the compressing in step (g) has a load of from about 10 to about 50 horsepower per metric kiloton per annum light olefins produced.

18. The process of claim 17, wherein the load is from about 15 to about 25 horsepower per metric kiloton per annum light olefins produced.

19. The process of claim 15, wherein the separating in step (b) forms a fuel oil stream, a gas oil stream, a gasoline stream, and the light olefins-containing stream, wherein the fuel oil stream contains at least a majority of the fuel oil that was present in the first effluent stream, wherein the gas oil stream contains at least a majority of the gas oil that was present in the first effluent stream, and wherein the gasoline stream contains at least a majority of the gasoline that was present in the first effluent stream.

20. The process of claim 15, wherein the quench overhead stream contains carbon dioxide, the process further comprising the step of:
(g) contacting at least a portion of the quench overhead stream with a caustic medium in a caustic wash unit under conditions effective to remove at least a portion of the carbon dioxide from the quench overhead stream.

21. The process of claim 15, wherein the process further comprises the step of:
(g) heating the quench bottoms stream in an oxygenate fractionation unit under conditions effective to form an oxygenate-containing overhead stream and a water-containing bottoms stream, wherein the oxygenate-containing overhead stream contains at least a majority of oxygenated components that were present in the quench bottoms stream, and wherein the water-containing bottoms stream contains at least a majority of the water that was present in the quench bottoms stream.

22. The process of claim 21, wherein the process further comprises the step of:
(h) directing at least a portion of the oxygenate-containing overhead stream to the methanol-to-olefin reactor for conversion to light olefins.

23. The process of claim 15, wherein the process further comprises the steps of:
(g) cooling a portion of the quench bottoms stream to form a cooled stream; and
(h) introducing the cooled stream into the quench unit to serve as the quench medium.

24. The process of claim 15, wherein step (d) occurs in the quench unit.

25. The process of claim 15, wherein step (d) occurs externally to the quench unit.

26. The process of claim 15, wherein the light olefins-containing stream contains at least 50 weight percent light olefins, based on the total weight of the light olefins-containing stream.

27. The process of claim 26, wherein the light olefins-containing stream contains at least 75 weight percent light olefins, based on the total weight of the light olefins-containing stream.

28. The process of claim 27, wherein the light olefins-containing stream contains at least 90 weight percent light olefins, based on the total weight of the light olefins-containing stream.

29. A process for forming light olefins, wherein the process comprises the steps of:
(a) contacting hydrocarbons with steam in a cracking furnace under first conditions effective to form a first effluent stream containing fuel oil, gas oil, gasoline, water and light olefins;
(b) separating the first effluent stream into a light olefins-containing stream and one or more heavy hydrocarbon streams, wherein the light olefins-containing stream contains at least a majority of the light olefins that were present in the first effluent stream;
(c) contacting at least a portion of the light olefins-containing stream with a first quench medium in a first quench unit under second conditions effective to form a first quench overhead stream and a first quench bottoms stream, wherein the first quench overhead stream contains at least a majority of the light olefins that were present in the at least a portion of the light olefins-containing stream, and wherein the first quench bottoms stream contains first condensed components and at least a majority of the water that was present in the at least a portion of the light olefins-containing stream;
(d) contacting methanol with a molecular sieve catalyst in a methanol-to-olefin reactor under third conditions effective to form a second effluent stream containing water and light olefins;
(e) contacting at least a portion of the second effluent stream with a second quench medium in a second quench unit under fourth conditions effective to form a second quench overhead stream and a second quench bottoms stream, wherein the second quench overhead stream contains at least a majority of the light olefins that were present in the at least a portion of the second effluent stream, and wherein the second quench bottoms stream contains second condensed components and at least a majority of the water that was present in the at least a portion of the second effluent stream;
(f) combining at least a portion of the first quench overhead stream with at least a portion of the second quench overhead stream to form a combined stream;
(g) compressing at least a portion of the combined stream to form a compressed stream; and
(h) fractionating at least a portion of the compressed stream to form a light olefins product stream and one or more C4+ hydrocarbon streams, wherein the light olefins product stream contains a majority of the light olefins that were present in the compressed stream.

30. The process of claim 29, wherein the compressing in step (g) has a load of from about 10 to about 50 horsepower per metric kiloton per annum light olefins produced.

31. The process of claim 30, wherein the load is from about 15 to about 25 horsepower per metric kiloton per annum light olefins produced.

32. The process of claim 29, wherein the separating in step (b) forms a fuel oil stream, a gas oil stream, a gasoline stream, and the light olefins-containing stream, wherein the fuel oil stream contains at least a majority of the fuel oil that was present in the first effluent stream, wherein the gas oil stream contains at least a majority of the gas oil that was present in the first effluent stream, and wherein the gasoline stream contains at least a majority of the gasoline that was present in the first effluent stream.

33. The process of claim 29, wherein the first quench overhead stream contains carbon dioxide, the process further comprising the step of:
(i) contacting at least a portion of the first quench overhead stream with a first caustic medium in a first caustic wash unit under fifth conditions effective to remove at least a portion of the carbon dioxide from the first quench overhead stream.

34. The process of claim 33, wherein the second quench overhead stream contains carbon dioxide, the process further comprising the step of:
(j) contacting at least a portion of the second quench overhead stream with a second caustic medium in a second caustic wash unit under sixth conditions effective to remove at least a portion of the carbon dioxide from the second quench overhead stream.

35. The process of claim 29, wherein the combined stream contains carbon dioxide, the process further comprising the step of:
(i) contacting at least a portion of the combined stream with a caustic medium in a caustic wash unit under fifth conditions effective to remove at least a portion of the carbon dioxide from the combined stream.

36. The process of claim 29, wherein the second quench overhead stream contains carbon dioxide, the process further comprising the step of:
(i) contacting at least a portion of the second quench overhead stream with a first caustic medium in a first caustic wash unit under fifth conditions effective to remove at least a portion of the carbon dioxide from the second quench overhead stream.

37. The process of claim 29, wherein the process further comprises the step of:
(i) heating the second quench bottoms stream in an oxygenate fractionation unit under conditions effective to form an oxygenate-containing overhead stream and a water-containing bottoms stream, wherein the oxygenate-containing overhead stream contains at least a majority of oxygenated components that were present in the second quench bottoms stream, and wherein the water-containing bottoms stream contains at least a majority of the water that was present in the second quench bottoms stream.

38. The process of claim 37, wherein the process further comprises the step of:
(j) directing at least a portion of the oxygenate-containing overhead stream to the methanol-to-olefin reactor for conversion to light olefins.

39. The process of claim 29, wherein the process further comprises the steps of:
(i) cooling a portion of the first quench bottoms stream to form a cooled stream; and
(j) introducing the cooled stream into the first quench unit to serve as the first quench medium.

40. The process of claim 29, wherein the process further comprises the steps of:
(i) cooling a portion of the second quench bottoms stream to form a cooled stream; and
(j) introducing the cooled stream into the second quench unit to serve as the second quench medium.

41. The process of claim 29, wherein the light olefins-containing stream contains at least 50 weight percent light olefins, based on the total weight of the light olefins-containing stream.

42. The process of claim 41, wherein the light olefins-containing stream contains at least 75 weight percent light olefins, based on the total weight of the light olefins-containing stream.

43. The process of claim 42, wherein the light olefins-containing stream contains at least 90 weight percent light olefins, based on the total weight of the light olefins-containing stream.

44. A process for forming light olefins, wherein the process comprises the steps of:
(a) contacting hydrocarbons with steam in a cracking furnace under first conditions effective to form a first effluent stream containing fuel oil, gas oil, gasoline and light olefins;
(b) separating the first effluent stream into a light olefins-containing stream and one or more heavy hydrocarbon streams, wherein the light olefins-containing stream contains at least a majority of the light olefins that were present in the first effluent stream;
(c) contacting at least a portion of the light olefins-containing stream with a first quench medium in a first quench unit under second conditions effective to form a first quench overhead stream and a first quench bottoms stream, wherein the first quench overhead stream contains at least a majority of the light olefins that were present in the at least a portion of the light olefins-containing stream, and wherein the first quench bottoms stream contains first condensed components and at least a majority of the water that was present in the at least a portion of the light olefins-containing stream;
(d) compressing at least a portion of the first quench overhead stream to form a first compressed stream;
(e) contacting methanol with a molecular sieve catalyst in a methanol-to-olefin reactor under third conditions effective to form a second effluent stream containing water and light olefins;
(f) contacting at least a portion of the second effluent stream with a second quench medium in a second quench unit under fourth conditions effective to form a second quench overhead stream and a second quench bottoms stream, wherein the second quench overhead stream contains at least a majority of the light olefins that were present in the at least a portion of the second effluent stream, and wherein the second quench bottoms stream contains second condensed components and at least a majority of the water that was present in the at least a portion of the second effluent stream;
(g) compressing at least a portion of the second quench overhead stream to form a second compressed stream;
(h) combining at least a portion of the first compressed stream and at least a portion of the second compressed stream to form a combined stream; and
(i) fractionating at least a portion of the combined stream to form a light olefins product stream and one or more C4+ hydrocarbon streams, wherein the light olefins product stream contains a majority of the light olefins that were present in the combined stream.

45. The process of claim 44, wherein the separating in step (b) forms a fuel oil stream, a gas oil stream, a gasoline stream, and the light olefins-containing stream, wherein the fuel oil stream contains at least a majority of the fuel oil that was present in the first effluent stream, wherein the gas oil stream contains at least a majority of the gas oil that was present in the first effluent stream, and wherein the gasoline stream contains at least a majority of the gasoline that was present in the first effluent stream.

46. The process of claim 44, wherein the first quench overhead stream contains carbon dioxide, the process further comprising the step of:
(j) contacting at least a portion of the first quench overhead stream with a first caustic medium in a first caustic wash unit under fifth conditions effective to remove at least a portion of the carbon dioxide from the first quench overhead stream.

47. The process of claim 46, wherein the second quench overhead stream contains carbon dioxide, the process further comprising the step of:
(k) contacting at least a portion of the second quench overhead stream with a second caustic medium in a second caustic wash unit under sixth conditions effective to remove at least a portion of the carbon dioxide from the second quench overhead stream.

48. The process of claim 44, wherein the combined stream contains carbon dioxide, the process further comprising the step of:
(j) contacting at least a portion of the combined stream with a caustic medium in a caustic wash unit under fifth conditions effective to remove at least a portion of the carbon dioxide from the combined stream.

49. The process of claim 44, wherein the second quench overhead stream contains carbon dioxide, the process further comprising the step of:
(j) contacting at least a portion of the second quench overhead stream with a first caustic medium in a first caustic wash unit under fifth conditions effective to remove at least a portion of the carbon dioxide from the second quench overhead stream.

50. The process of claim 44, wherein the process further comprises the step of:
(j) heating the second quench bottoms stream in an oxygenate fractionation unit under conditions effective to form an oxygenate-containing overhead stream and a water-containing bottoms stream, wherein the oxygenate-containing overhead stream contains at least a majority of oxygenated components that were present in the second quench bottoms stream, and wherein the water-containing bottoms stream contains at least a majority of the water that was present in the second quench bottoms stream.

51. The process of claim 50, wherein the process further comprises the step of:
(k) directing at least a portion of the oxygenate-containing overhead stream to the methanol-to-olefin reactor for conversion to light olefins.

52. The process of claim 44, wherein the process further comprises the steps of:
(j) cooling a portion of the first quench bottoms stream to form a cooled stream; and
(k) introducing the cooled stream into the first quench unit to serve as the first quench medium.

53. The process of claim 44, wherein the process further comprises the steps of:
(j) cooling a portion of the second quench bottoms stream to form a cooled stream; and
(k) introducing the cooled stream into the second quench unit to serve as the second quench medium.

54. The process of claim 44, wherein the light olefins-containing stream contains at least 50 weight percent light olefins, based on the total weight of the light olefins-containing stream.

55. The process of claim 54, wherein the light olefins-containing stream contains at least 75 weight percent light olefins, based on the total weight of the light olefins-containing stream.

56. The process of claim 55, wherein the light olefins-containing stream contains at least 90 weight percent light olefins, based on the total weight of the light olefins-containing stream.

57. A system for forming light olefins, comprising:
(a) a steam cracking furnace having one or more inlets for receiving hydrocarbons and steam, and a first outlet line for carrying a first product stream;
(b) a methanol to olefin conversion unit having one or more inlets for receiving a methanol-containing feedstock, and a second outlet line for carrying a second product stream;
(c) a quench unit in fluid communication with the first outlet line and the second outlet line, wherein the quench unit includes an overhead line, a condensate line and a pumparound stream, wherein the pumparound stream is arranged to deliver condensed components from a lower region of the quench unit to an upper region of the quench unit;
(d) a compressor having a compressor inlet and a compressor outlet, wherein the compressor inlet is in fluid communication with the overhead line; and
(e) a fractionation system having a fractionator inlet, a light olefin outlet line and one or more C4+ hydrocarbon outlet lines, wherein the fractionator inlet is in fluid communication with the compressor outlet.

58. A system for forming light olefins, comprising:
(a) a steam cracking furnace having one or more inlets for receiving hydrocarbons and steam, and a first outlet line for carrying a first product stream;
(b) a first quench unit in fluid communication with the first outlet line, wherein the first quench unit includes a first overhead line, a first condensate line and a first pumparound stream, wherein the first pumparound stream is arranged to deliver first condensed components from a first lower region of the first quench unit to a first upper region of the first quench unit;
(c) a methanol to olefin conversion unit having one or more inlets for receiving a methanol-containing feedstock, and a second outlet line for carrying a second product stream;
(d) a second quench unit in fluid communication with the second outlet line, wherein the second quench unit includes a second overhead line, a second condensate line and a second pumparound stream, wherein the second pumparound stream is arranged to deliver second condensed components from a second lower region of the second quench unit to a second upper region of the second quench unit;
(e) a compressor having a compressor inlet and a compressor outlet, wherein the compressor inlet is in fluid communication with the first overhead line and the second overhead line; and
(f) a fractionation system having a fractionator inlet, a light olefin outlet line and one or more C4+ hydrocarbon outlet lines, wherein the fractionator inlet is in fluid communication with the compressor outlet.

59. A system for forming light olefins, comprising:
(a) a steam cracking furnace having one or more inlets for receiving hydrocarbons and steam, and a first outlet line for carrying a first product stream;
(b) a first quench unit in fluid communication with the first outlet line, wherein the first quench unit includes a first overhead line, a first condensate line and a first pumparound stream, wherein the first pumparound stream is arranged to deliver first condensed components from a first lower region of the first quench unit to a first upper region of the first quench unit;
(c) a first compressor having a first compressor inlet and a first compressor outlet, wherein the first compressor inlet is in fluid communication with the first overhead line;
(d) a methanol to olefin conversion unit having one or more inlets for receiving a methanol-containing feedstock, and a second outlet line for carrying a second product stream;
(e) a second quench unit in fluid communication with the second outlet line, wherein the second quench unit includes a second overhead line, a second condensate line and a second pumparound stream, wherein the second pumparound stream is arranged to deliver second condensed components from a second lower region of the second quench unit to a second upper region of the second quench unit;
(f) a second compressor having a second compressor inlet and a second compressor outlet, wherein the second compressor inlet is in fluid communication with the second overhead line; and
(g) a fractionation system having a fractionator inlet, a light olefin outlet line and one or more C4+ hydrocarbon outlet lines, wherein the fractionator inlet is in fluid communication with the first compressor outlet and the second compressor outlet.
